(19) United States
(12) Patent Application Publication
(10) Pub. No.: US 2018/0087114 A1
(43) Pub. Date: Mar. 29, 2018

(54) EARLY ASSESSMENT OF MECHANISM OF ACTION AND EFFICACY OF ANTI-CANCER THERAPIES USING MOLECULAR MARKERS IN BODILY FLUID

(71) Applicant: Trovagene, Inc., San Diego, CA (US)

(72) Inventors: Vlada Melnikova, San Diego, CA (US); Mark G. Erlander, San Diego, CA (US)

(73) Assignee: Trovagene, Inc., San Diego, CA (US)

(21) Appl. No.: 15/555,236

(22) PCT Filed: Mar. 4, 2016
(86) PCT No.: PCT/US16/20967

§ 371 Date: Sep. 1, 2017

Related U.S. Application Data
(60) Provisional application No. 62/128,982, filed on Mar. 5, 2015.
(60) Provisional application No. 62/232,585, filed on Sep. 25, 2015.

(51) Int. Cl. C12Q 1/68

(52) U.S. Cl. CPC C12Q 1/6886; C12Q 2600/106; C12Q 2600/156; C12Q 2600/118

(57) ABSTRACT

Provided is a method of determining responsiveness of a subject to a treatment for a cancer. Also provided is a method of determining treatment recommendations for a subject with cancer. Additionally provided is a method of treating a subject with cancer.

### CROSS-REFERENCE TO RELATED APPLICATIONS

This application claims the benefit of U.S. Provisional Application No. 62/128,982, filed Mar. 5, 2015, and U.S. Provisional Application No. 62/232,585, filed Sep. 25, 2015, both incorporated by reference herein in their entirety.

### BACKGROUND OF THE INVENTION

### (1) Field of the Invention

The present application generally relates to the use of biomarkers in cancer diagnosis. More specifically, the application relates to the use of changes in cancer biomarker presence in bodily fluids before and during treatment to assess treatment efficacy.

### (2) Description of the Related Art

The related art is discussed in the cited references.

### BRIEF SUMMARY OF THE INVENTION

The present invention is based on the discovery that, when a subject is being treated for a cancer, various effects of the treatment, including early detection of resistance to therapy, mechanism of action, and early evidence of responsiveness, can be determined by measuring the quantity of a mutation characteristic of the cancer in a plurality of samples of a bodily fluid of the subject taken at different time points after administration of the treatment.

Thus, provided herein is a method comprising quantifying a mutation in nucleic acid fragments in a plurality of samples of a bodily fluid of a subject, each sample taken at a different time point after the subject begins a treatment. In this method, the mutation is associated with a cancer in the subject, and the treatment is against the cancer.

Also provided is a method of determining treatment recommendations for a subject with cancer. The method comprises determining expected progression-free survival, expected objective response, and/or expected overall survival of the subject by the method described above, and (a) recommending continuation of the treatment if expected progression-free survival, expected objective response, and/or expected overall survival is favorable, or (b) recommending a change of treatment if expected progression-free survival, expected objective response, and/or expected overall survival is unfavorable.

Additionally provided is a method of determining treatment recommendations for a subject with cancer. The method comprises determining responsiveness of the subject by the above-described method, and (a) recommending continuation of the treatment if a spike in the quantity of the mutation was present within one week of starting the treatment, or (b) recommending a change of treatment if a spike in the quantity of the mutation was not present within one week of starting the treatment.

Further provided is a method of treating a subject with cancer. The method comprises determining expected progression-free survival, expected objective response, and/or expected overall survival of the subject by any of the above-described methods, and (a) continuing the treatment if expected progression-free survival, expected objective response, and/or expected overall survival is favorable, or (b) changing the treatment if expected progression-free survival, expected objective response, and/or expected overall survival is unfavorable.

In additional embodiments, another method of treating a subject with cancer is provided. The method comprises determining responsiveness of the subject by any of the above-described methods that measure a spike in mutant gene levels, and (a) continuing the treatment if a spike in the quantity of the mutation was present within one week of starting the treatment, or (b) changing the treatment if a spike in the quantity of the mutation was not present within one week of starting the treatment.

### BRIEF DESCRIPTION OF THE DRAWINGS

FIG. 1 is graphs showing results of urine testing of non-small cell lung carcinoma patients that were monitored for early acquisition of the EGFR T790M mutation.

FIG. 2 is graphs showing results of urine testing of non-small cell lung carcinoma patients that were monitored for early acquisition of the EGFR T790M mutation.

FIG. 3 is graphs showing results of urine testing of non-small cell lung carcinoma patients that were monitored for early acquisition of the EGFR T790M mutation as well as the response to treatment.

FIG. 4 is graphs showing results of urine testing of non-small cell lung carcinoma patients that were monitored for early acquisition of the EGFR T790M mutation as well as the response to treatment.

FIG. 5 is graphs showing results of urine testing of non-small cell lung carcinoma patients that were monitored for early acquisition of the EGFR T790M mutation as well as the response to treatment.

FIGS. 6A and 6B are graphs showing urine monitoring of EGFR T790 and EGFR Exon 19del mutations in a lung cancer patient along with the CT scan results, measured as the sum of the longest diameters of the lesions (in FIG. 6A).

FIGS. 7A and 7B are graphs showing urine monitoring of EGFR T790 and L858R mutations in a lung cancer patient along with the CT scan results, measured as the sum of the longest diameters of the lesions (in FIG. 7A).

FIGS. 8A and 8B are graphs showing urine monitoring of EGFR T790 and EGFR Exon 19del mutations in a lung cancer patient along with the CT scan results, measured as the sum of the longest diameters of the lesions (in FIG. 8A).

FIGS. 9A and 9B are graphs showing urine monitoring of EGFR T790 and EGFR Exon 19del mutations in a lung cancer patient along with the CT scan results, measured as the sum of the longest diameters of the lesions (in FIG. 9A).

FIGS. 10A and 10B are graphs showing urine monitoring of EGFR T790 and L858R mutations in a lung cancer patient along with the CT scan results, measured as the sum of the longest diameters of the lesions (in FIG. 10A).

FIG. 11A shows EGFR T790M levels for 9 weeks monitored at baseline, week 1 and week 2. FIGS. 11B, 11C and 11D are models of EGFR T790M occurrence in urine before and during drug treatment over several months with a partial response then later failure of the therapy. FIG. 11B shows a model of a responsive treatment over 15 months; FIG. 11C shows the model over the first week; and FIG. 11D shows both typical responsive and non-responsive treatments.

FIGS. 12A, 12B and 12C are graphs showing quantification of EGFR Mutant and Wild-Type DNA Blends by PCR-NGS. FIG. 12A shows the analysis of a dilution series of indicated mutant EGFR variants spiked into 60 ng (≈18,180 genome equivalents) of WT DNA. Each data point represents one preparative within 6 independent dilutions series prepared and analyzed by two operators on two different instruments on three non-consecutive days for a total of 18 samples per dilution point. An analysis algorithm was applied to transform the mutant EGFR sequencing reads into the absolute mutant copies detected. The box-and-whisker plots show the median (center line), 25^{th} and 75^{th} percentiles (box) with the connecting “whiskers” extending from the first quartile minus 1.5 of the interquartile range (IQR, the third quartile less the first quartile) and the third quartile plus 1.5 of the IQR. A positive Spearman's correlation close to 1 indicates a strong, positive relationship between the absolute mutant EGFR copies detected and the absolute mutant EGFR copies per input. FIG. 28B shows inter-run reproducibility of the EGFR exon 19 deletions, L858R and T790M enrichment PCR-NGS assays for the dilution series shown in FIG. 12A. The Coefficient of Variation Percent (CV %) was calculated as the ratio of the standard deviation to the mean of the absolute EGFR copies detected within each absolute copy per input level and is reported as a percentage.

FIGS. 13A, 13B, 13C, and 13D are graphs showing quantification of EGFR mutation levels in urine of patients with NSCLC before and after 1 and 2 weeks of osimertinib therapy. Urine samples were collected from patients prior to osimertinib treatment and at week 1 or around week 2 time point on treatment. T790M ctDNA and corresponding EGFR L858R or exon 19 deletion levels shown as copies per 100,000 genome equivalents (FIGS. 29A, B) or as percent of respective baselines (FIGS. 13C, D). A significant relative decrease in T790M mutation signal from baseline was observed at week 1 and 2 on treatment (one-sided p-values of 0.014 and 0.045, respectively, using Wilcoxon's Test) (FIG. 13C). Similar patterns were observed for the activating mutations L858R, exon 19 deletions at week 1 and week 2.

FIG. 14 is graphs showing daily dynamics of ctDNA EGFR mutation levels on osimertinib therapy. Urine samples were collected from patients prior to osimertinib treatment at baseline and daily on treatment. A consistent pattern of an overall decrease in the numbers of copies between baseline to day 7 with intermittent peaks distributed over the first week was observed. Data points are mutant EGFR copies per 100,000 genome equivalents detected. Dashed lines indicate clinical detection cut-offs for the EGFR activating mutations.

FIG. 15 is a graph showing results of urine and plasma testing of KRAS ctDNA in a colorectal cancer (CRC) patient who underwent curative intent surgery during the monitoring.

FIGS. 16A, 16B, 16C and 16D are graphs showing results of urine and plasma testing of KRAS ctDNA in colorectal cancer (CRC) patients who underwent incomplete, palliative surgery during the monitoring.

FIGS. 17A and 17B are graphs showing urine KRAS G13D monitoring in urine along with carcinoembryonic antigen (CEA) monitoring in plasma (FIG. 17A) and urine and plasma monitoring of KRAS G13D in a colorectal cancer (CRC) patient (FIG. 17B).

FIGS. 18A and 18B are graphs showing urine KRAS G13D monitoring in urine along with CEA monitoring in plasma (FIG. 18A) and urine and plasma monitoring of KRAS G13D in a CRC patient (FIG. 18B).

FIGS. 19A and 19B are graphs showing urine KRAS G12D monitoring in urine along with CEA monitoring in plasma (FIG. 19A) and urine and plasma monitoring of KRAS G12D in a CRC patient (FIG. 19B).

FIGS. 20A and 20B are graphs showing urine KRAS G12D and G12S monitoring in urine along with CEA monitoring in plasma (FIG. 20A) and urine and plasma monitoring of KRAS G12D in a CRC patient (FIG. 20B).

FIG. 21 is an illustration showing the design of the study described in Example 5.

FIG. 22 is an illustration showing a significant association between baseline KRAS ctDNA levels and overall survival in pancreatic cancer.

FIG. 23 is a graph with Kaplan-Meier survival plots showing a significant association between baseline KRAS copies and overall survival.

FIG. 24 is an illustration showing the ability of combination KRAS determination and CA-19-9 determination in predicting overall survival in pancreatic cancer.

FIG. 25 is a graph with Kaplan-Meier survival plots of categories of results of KRAS and CA-19-9 determinations.

FIG. 26 is an illustration showing the effectiveness in utilizing KRAS determinations at baseline and at two weeks in predicting overall survival.

FIG. 27 is graphs showing that the longitudinal dynamics of KRAS ctDNA burden after two weeks of chemotherapy correlates with overall survival better than baseline KRAS.

FIG. 28 is graphs showing that the longitudinal dynamics of KRAS ctDNA burden after two weeks of chemotherapy correlates with overall survival better than baseline KRAS.

FIG. 29 is a graph showing that the longitudinal dynamics of KRAS ctDNA burden after two weeks of chemotherapy correlates with overall survival better than baseline KRAS.

### DETAILED DESCRIPTION OF THE INVENTION

As used herein, the singular forms “a”, “an” and “the” are intended to include the plural forms as well, unless the context clearly indicates otherwise. Additionally, the use of “or” is intended to include “and/or”, unless the context clearly indicates otherwise.

As used herein, the term “sample” refers to anything which may contain an analyte for which an analyte assay is desired. In many cases, the analyte is a cf nucleic acid molecule, such as a DNA, RNA or cDNA molecule encoding all or part of EGFR. The sample may be a biological sample, such as a biological fluid or a biological tissue. Examples of biological fluids include urine, blood, plasma, serum, saliva, semen, stool, sputum, cerebrospinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein, a “subject” includes a mammal. The mammal can be any mammal, e.g., a human, primate, mouse, rat, fowl, dog, cat, cow, horse, goat, camel, sheep or a pig. These methods can be applied to non-mammalian animals, e.g., birds, as well. In many cases, the subject is a human being.

The present invention is based on the discovery that, when a subject is being treated for a cancer or other diseases such as chronic viral, bacterial, parasitic or other pathogen infections, or transplant rejection, the effect of the treatment on the cancer or other disease can be predicted by measuring the quantity of a mutation characteristic of the cancer in a plurality of samples of a bodily fluid of the subject taken at different time points after administration of the treatment. Sampling within short time intervals, e.g., hours or days, provides significant information for determining efficacy and prognostic parameters such as described in Eisenhauer et al., 2009, in revised RECIST guidelines, e.g., complete response (CR), partial response (PR), progressive disease (PD), stable disease (SD), progression-free survival (PFS), time to progression (TTP), time to treatment failure (TTF), event-free survival (EPS), overall response rate (ORR), duration of response (DOR), objective response rate (ORR) as well as drug dosage assessment, and mechanism of action.

Thus, provided herein is a method comprising quantifying a mutation in nucleic acid fragments in a plurality of samples of a bodily fluid of a subject, each sample taken at a different time point after the subject begins a treatment. In this method, the mutation is associated with a cancer in the subject, and the treatment is against the cancer.

In these methods, the samples can be taken at any time in relation to the beginning of the treatment. In some embodiments, a sample is taken prior to, or at, the beginning of the treatment. In other embodiments, a sample is taken at least twice within seven days after administration of the treatment. In additional embodiments, a sample is taken within about 1 hour, 4 hours, 8 hours, 12 hours and/or 24 hours, after the beginning of treatment. In other embodiments, a sample is taken daily for seven days after beginning the treatment. As shown in the Examples, information within the first week after treatment begins, or after surgery, provides valuable information relating to, e.g., response to the treatment or surgery.

In additional embodiments, a sample is taken prior to, or at, the beginning of the treatment, and at least at once within 3 weeks after beginning the treatment. As shown, e.g., in Example 4, responsiveness to treatment is detectable as early as two weeks after beginning the treatment.

As shown in the Examples, there is often a significant drop in the mutation quantity in urine within 4-24 hours after the beginning of treatment. This is often followed by a significant increase followed by a significant decrease (“spike”) of the mutation within 7 days of treatment administration. Such determinations have predictive value, as demonstrated in the Example.

Non-limiting examples of characteristics that the temporal variation in quantity of the mutation among the plurality of samples is used to determine include (a) a mechanism of treatment action, (b) dosing information, (c) responsiveness, (d) expected progression-free survival, (e) expected objective response, and/or (f) expected overall survival. The rapid determination of these parameters helps not only a cancer patient, but also in clinical trials of drugs, since these methods would shorten the time that these parameters can be determined for the drug in question, potentially saving time and reducing costs for those trials. See, e.g., Example 3.

Mechanism of Drug Action.

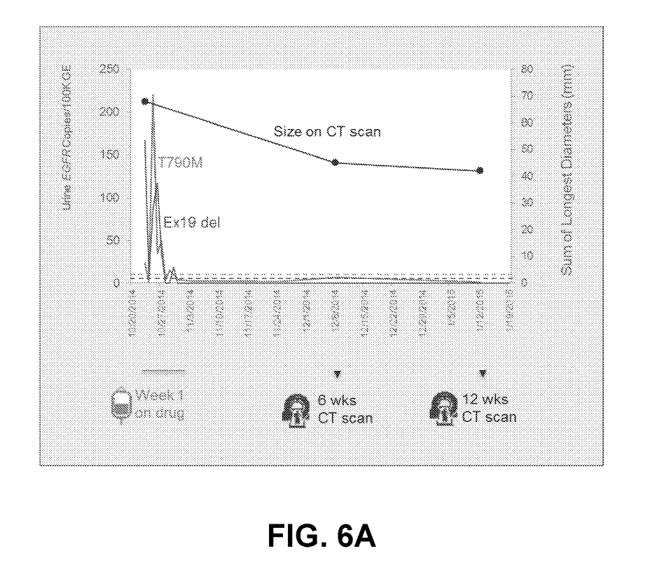

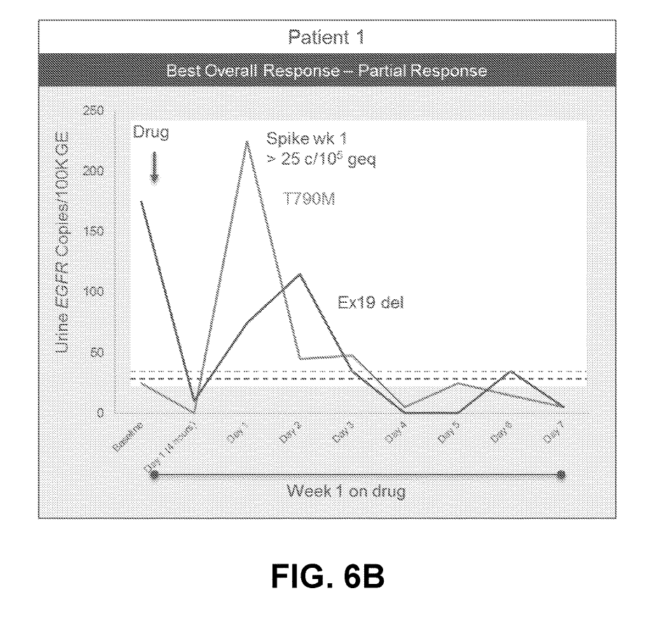

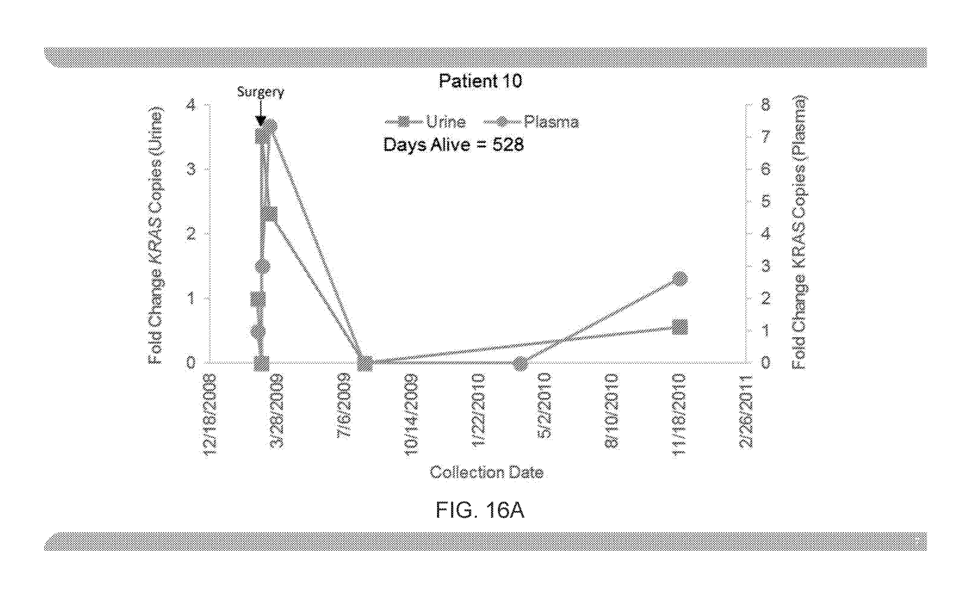

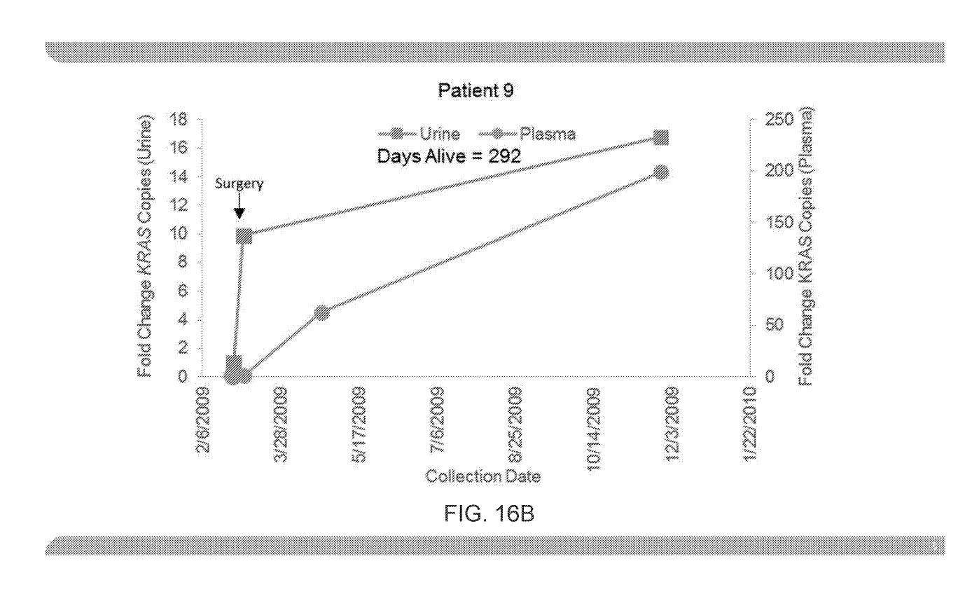

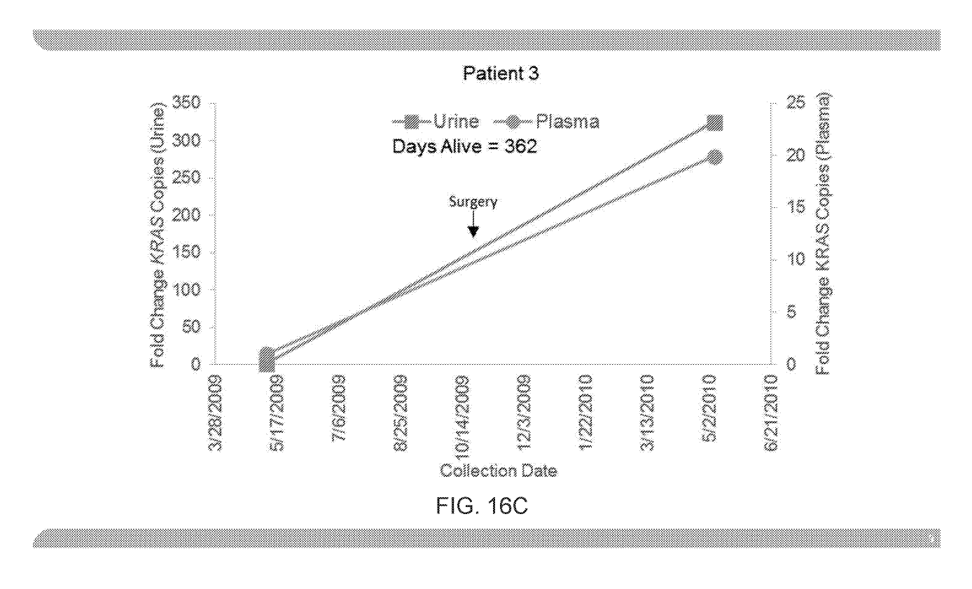

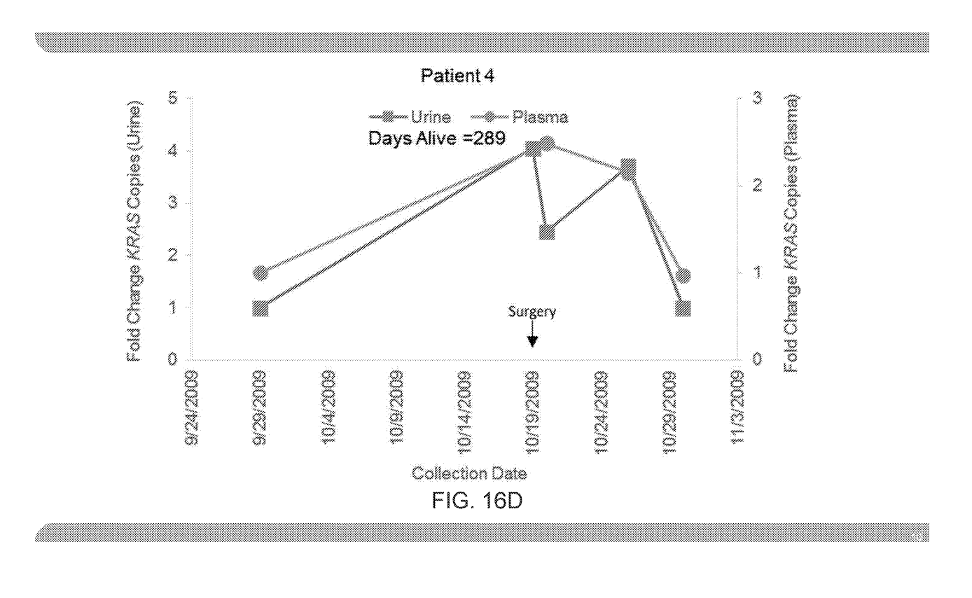

A temporal assessment of levels of the cancer mutation, if done early enough after treatment, can provide information as to the mechanism of cancer cell death induced by the treatment. Since apoptosis is a programed process that takes 4 to 48 hours, a more immediate increase of mutant nucleic acid into bodily fluids indicates cell death by another mechanism, e.g., cell disruption. See Example 2, where patients treated with a tyrosine kinase inhibitor had an initial dip in the amount of the cancer mutation before experiencing a spike in the mutation quantity in about one day (e.g., FIG. 6B), indicating apoptosis. Compare with Example 4 and FIG. 16A, showing monitoring of urine and plasma in a colorectal cancer patients for a KRAS mutation, where surgery was palliative. Immediately after surgery (second time point) there was a large amount of DNA with the monitored KRAS mutation, indicating that the quick release of mutant DNA was due to cell disruption from the surgery, not apoptosis, since mutant DNA resulting from apoptosed cells would not be expected to appear in bodily fluids so quickly.

Dosing Information.

The determination of levels of the cancer mutation early in a treatment can assist in the determination of a proper dosage level of a medication. An early response that is less than expected based on historical data or comparison with control and standard samples with known responses may indicate that a higher dosage is needed. In this way, a dose can be titrated for each individual. This information is particularly useful when the medication is in clinical trials, since efficacious dosage ranges can be established much more quickly than without the ability to quickly assess efficacy that these methods enable.

Responsiveness.

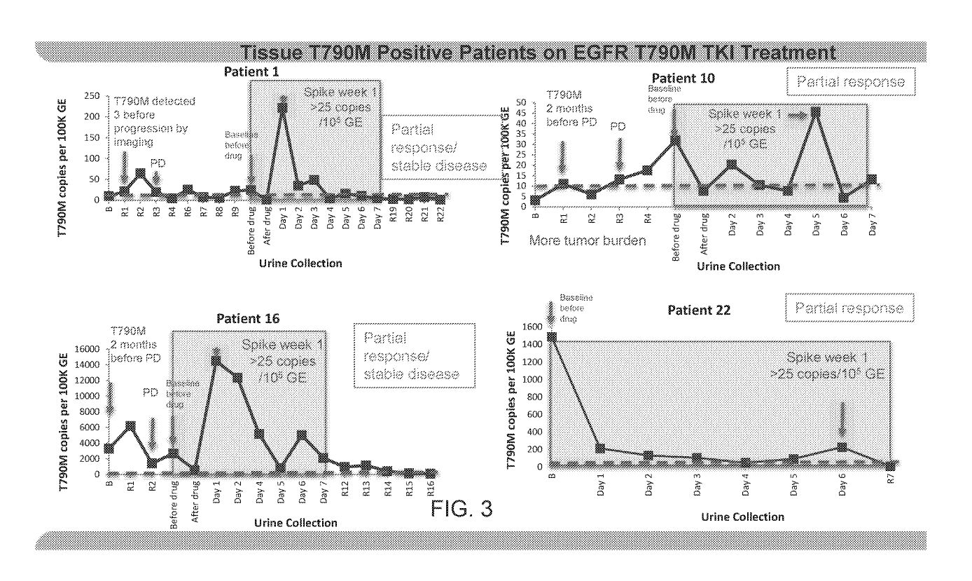

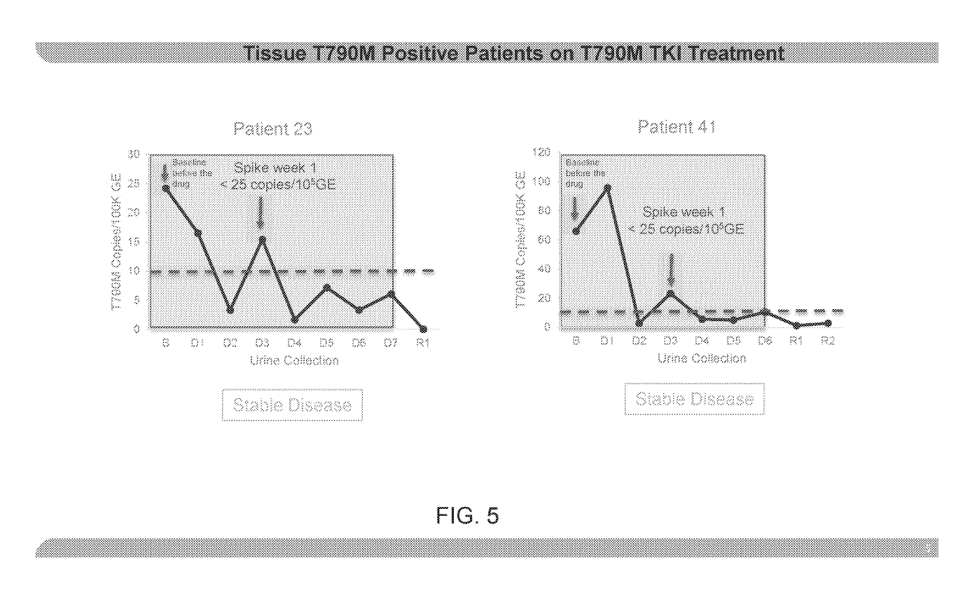

As shown in, e.g., in FIGS. 3-5 and 7, a large spike (e.g., increase greater than about 25, 50 or 100 copies of the mutation per 10⁵ genome equivalents [“GE”] followed by a decrease to less than 10 copies per 10⁵ GE within about a week after treatment indicates responsiveness. The skilled artisan can develop models for predicting expected progression-free survival, expected objective response, expected overall survival or any other parameters (e.g., CR, PR, PD, SD, TTP, TTF, EPS, ORR, or DOR) without undue experimentation by simply comparing the pharmacodynamics of the mutation in a bodily fluid with the pharmacodynamics of patients with known outcomes. The rapid establishment of those clinical parameters are not only useful for individual patients, but also in determining the efficacy of a drug in clinical trials.

In some of these embodiments, the presence of a significant increase followed by a significant decrease (“spike”) of the mutation within 7 days of administration of the treatment indicates responsiveness.

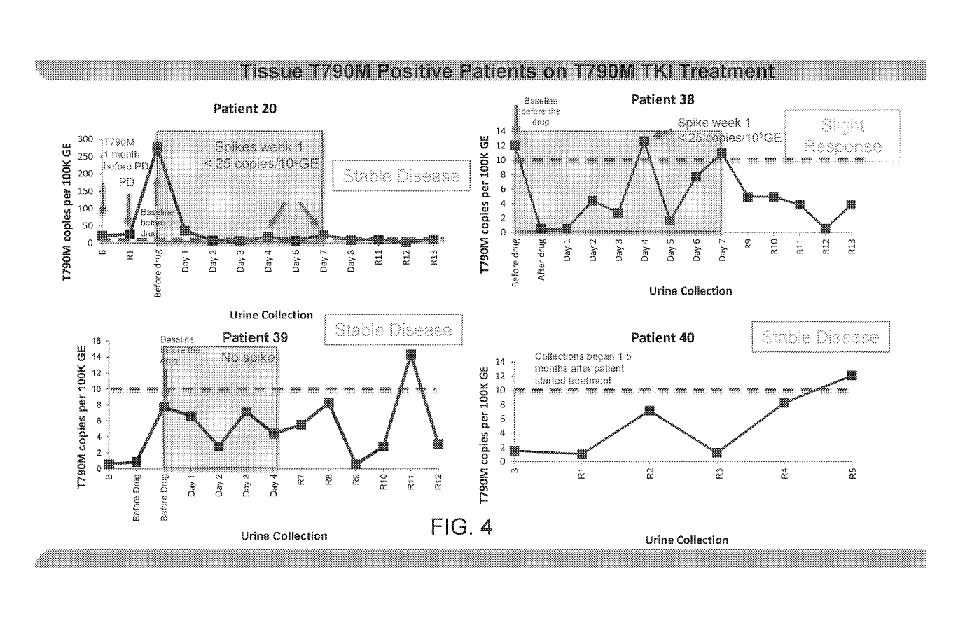

In various embodiments of this method, the absence of a spike, or low spikes, e.g., below 100, 50 or 25 copies per 10⁵ GE indicates stable disease with the treatment. See, e.g., FIGS. 4, 5 and 13.

In additional embodiments, the significant increase is to greater than about 25, 50 or 100 copies of the mutation per 10⁵ genome equivalents (“GE”). See Examples. In further embodiments, the significant decrease is to below about 10 copies of the mutation per 10⁵ GE.

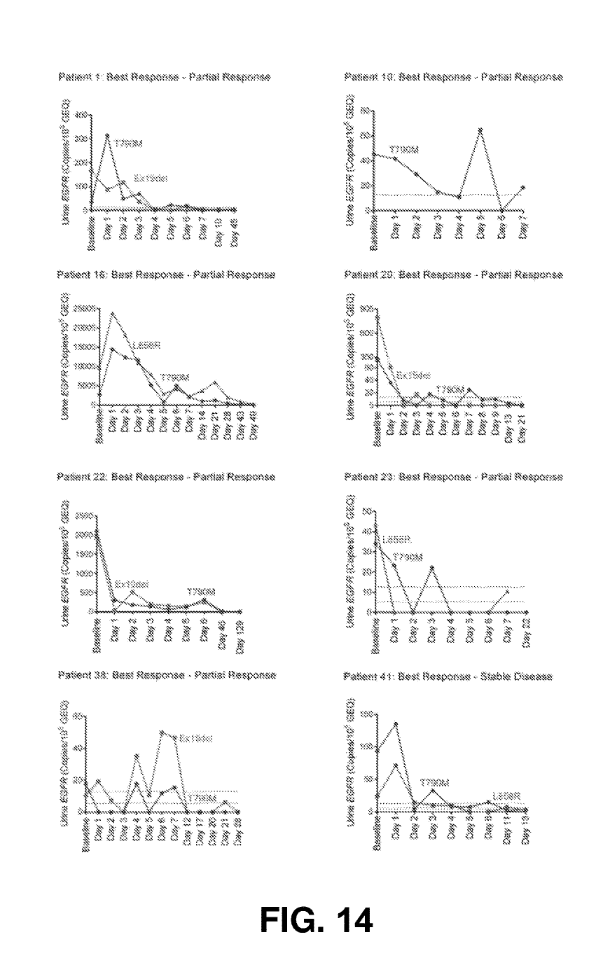

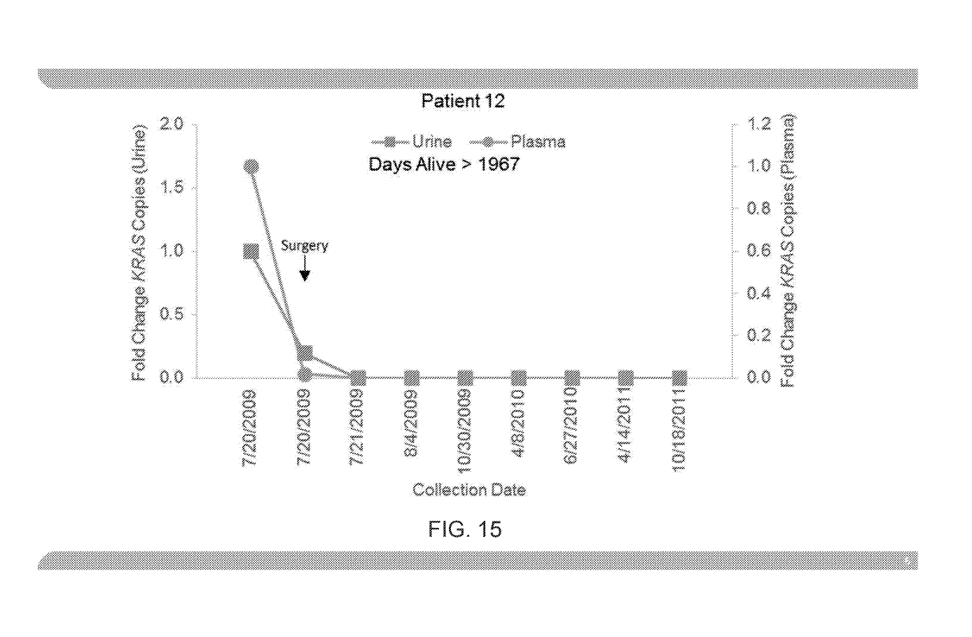

These methods can be applied to “resistance mutations” that are acquired after a first treatment for a cancer with a different mutation. An example of such a mutation is EGFR T790M, which is known to arise after treatment with first-line therapy against lung cancer with a different EGFR mutation. The present methods can also be used to monitor minimal residual disease to identify a resistance mutation before the relapse can be detected clinically, or to monitor the steady state of a responsive treatment over months or years. See FIGS. 15-20.

As shown in Example 3 and FIG. 13, responsiveness can be detected within two weeks. The patients in FIG. 13 that did not have a reduction in cancer mutation to less than about 25% of the baseline level within two weeks of the start of treatment did not respond to the treatment. Thus, using the present methods, the efficacy of a treatment can be determined within two weeks with a blood or urine test.

The methods are not narrowly limited to any particular kind of mutation that is associated with the cancer. In some embodiments, the mutation associated with the cancer is a point mutation or a rearrangement.

These methods are also not narrowly limited to use with a cancer associated with a mutation in any particular gene. In various embodiments, the mutation associated with the cancer is in an a point mutation in an ABL1, BRAF, CHEK1, FANCC, GATA3, JAK2, MITF, PDCD1LG2, RBM10, STAT4, ABL2, BRCA1, CHEK2, FANCD2, GATA4, JAK3, MLH1, PDGFRA, RET, STK11, ACVR1B, BRCA2, CIC, FANCE, GATA6, JUN, MPL, PDGFRB, RICTOR, SUFU, AKT1, BRD4, CREBBP, FANCF, GID4(C17orf39), KAT6A (MYST3), MRE11A, PDK1, RNF43, SYK, AKT2, BRIP1, CRKL, FANCG, GLI1, KDMSA, MSH2, PIK3C2B, ROS1, TAF1, AKT3, BTG1, CRLF2, FANCL, GNA11, KDMSC, MSH6, PIK3CA, RPTOR, TBX3, ALK, BTK, CSF1R, FAS, GNA13, KDM6A, MTOR, PIK3CB, RUNX1, TERC, AMER1 (FAM123B), C11orf30 (EMSY), CTCF, FAT1, GNAQ, KDR, MUTYH, PIK3CG, RUNX1T1, TERT promoter, APC, CARD11, CTNNA1, FBXW7, GNAS, KEAP1, MYC, PIK3R1, SDHA, TET2, AR, CBFB, CTNNB1, FGF10, GPR124, KEL, MYCL (MYCL1), PIK3R2, SDHB, TGFBR2, ARAF, CBL, CUL3, FGF14, GRIN2A, KIT, MYCN, PLCG2, SDHC, TNFAIP3, ARFRP1, CCND1, CYLD, FGF19, GRM, 3 KLHL6, MYD88, PMS2, SDHD, TNFRSF14, ARID1A, CCND2, DAXX, FGF23, GSK3B, KMT2A (MLL), NF1, POLD1, SETD2, TOP1, ARID1B, CCND3, DDR2, FGF3, H3F3A, KMT2C (MLL3), NF2, POLE, SF3B1, TOP2A, ARID2, CCNE1, DICER1, FGF4, HGF, KMT2D (MLL2), NFE2L2, PPP2R1A, SLIT2, TP53, ASXL1, CD274, DNMT3A, FGF6, HNF1A, KRAS, NFKBIA, PRDM1, SMAD2, TSC1, ATM, CD79A, DOT1L, FGFR1, HRAS, LMO1, NKX2-1, PREX2, SMAD3, TSC2, ATR, CD79B, EGFR, FGFR2, HSD3B1, LRP1B, NOTCH1, PRKAR1A, SMAD4, TSHR, ATRX, CDC73, EP300, FGFR3, HSP9OAA1, LYN, NOTCH2, PRKCI, SMARCA4, U2AF1, AURKA, CDH1, EPHA3, FGFR4, IDH1, LZTR1, NOTCH3, PRKDC, SMARCB1, VEGFA, AURKB, CDK12, EPHA5, FH, IDH2, MAGI2, NPM1, PRSS8, SMO, VHL, AXIN1, CDK4, EPHA7, FLCN, IGF1R, MAP2K1, NRAS, PTCH1, SNCAIP, WISPS, AXL, CDK6, EPHB1, FLT1, IGF2, MAP2K2, NSD1, PTEN, SOCS1, WT1, BAP1, CDK8, ERBB2, FLT3, IKBKE, MAP2K4, NTRK1, PTPN11, SOX10, XPO1, BARD1, CDKN1A, ERBB3, FLT4, IKZF1, MAP3K1, NTRK2, QKI, SOX2, ZBTB2, BCL2, CDKN1B, ERBB4, FOXL2, IL7R, MCL1, NTRK3, RAC1, SOX9, ZNF217, BCL2L1, CDKN2A, ERG, FOXP1, INHBA, MDM2, NUP93, RAD50, SPEN, ZNF703, BCL2L2, CDKN2B, ERRFI1, FRS2, INPP4B, MDM4, PAK3, RAD51, SPOP, BCL6, CDKN2C, ESR1, FUBP1, IRF2, MED12, PALB2, RAF1, SPTA1, BCOR, CEBPA, EZH2, GABRA6, IRF4, MEF2B, PARK2, RANBP2, SRC, BCORL1, CHD2, FAM46C, GATA1, IRS2, MEN1, PAX5, RARA, STAG2, BLM, CHD4, FANCA, GATA2, JAK1, MET, PBRM1, RB1, or STATS gene, or a rearrangement in an ALK, BRAF, BRD4, ETV4, FGFR1, KIT, MYC, NTRK2, RARA, TMPRSS2, BCL2, BRCA1, EGFR, ETV5, FGFR2, MSH2, NOTCH2, PDGFRA, RET, BCR, BRCA2, ETV1, ETV6, FGFR3, MYB, NTRK1, RAF1, or ROS1 gene. In certain of these embodiments, the mutation associated with the cancer is in an APC, ALK, BRAF, CDK4, CTNNB1, EGFR, FGFR1, FGFR2, FGFR3, HER3, PDGFRA, PDGFRB, AKT1, ESR1, AR, EZH2, FLT3, HER2, IDH1, IDH2, JAK2, KIT, KRAS, c-Myc, MEK1, NOTCH1, NRAS, PIK3CA, PTEN, SNV, TP53, CDKN2A, or RB1 gene.

In some more specific embodiments, the mutation associated with the cancer is in the EGFR gene, e.g., an EGFR activing mutation (e.g., Exon 19 deletions, Exon 21 L858R, Exon 21 L861Q, and others known in the art), or EGFR T790M. In various embodiments, the EGFR mutation is associated with a lung cancer.

In other embodiments, the mutation associated with the cancer is in the KRAS gene, e.g., KRAS G12D, G12S, or G13D. In some embodiments, the KRAS mutation is associated with colorectal cancer.

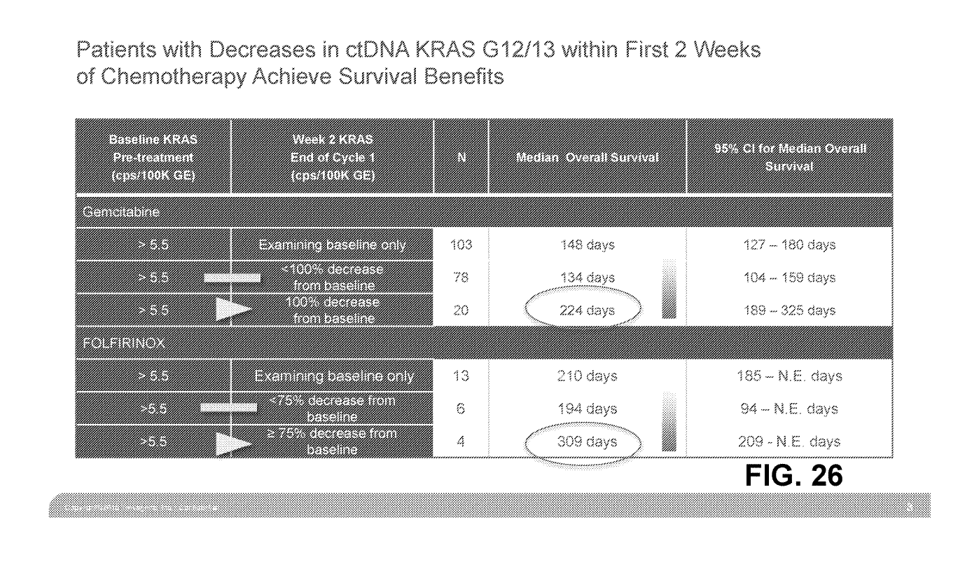

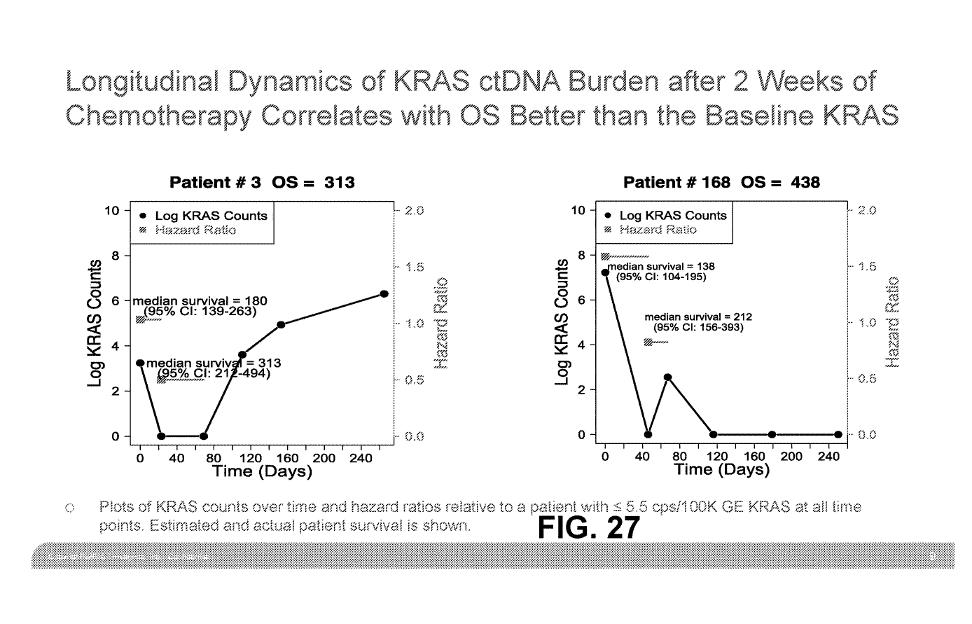

As further established in Example 5 below, the baseline level of a cancer gene, e.g., mutant KRAS, before treatment is useful for estimating overall survival, e.g., with pancreatic cancer. A more accurate estimate can be made if the baseline level is compared to the level two weeks after the start of therapy, where a large decrease indicates longer survival than a small decrease. For example, FIG. 26 shows that a decrease of 100% (i.e., not detectable) at two weeks indicates longer survival than a decrease of less than 100% when gemcitabine is used on pancreatic cancer, and a decrease of 75% or greater at two weeks indicates longer survival than a decrease of less than 75% when FOLFIRINOX is used on pancreatic cancer. Thus, the percentage can vary (e.g., 90%, 80%, 75%, 70%, 60%, 50%, 40%, 30%, 25%, 20%, 10%, or any value in between) depending on the cancer treatment, and the length of overall survival desired in the long vs. short survival group, and can be determined empirically without undue experimentation with any cancer-treatment combination.

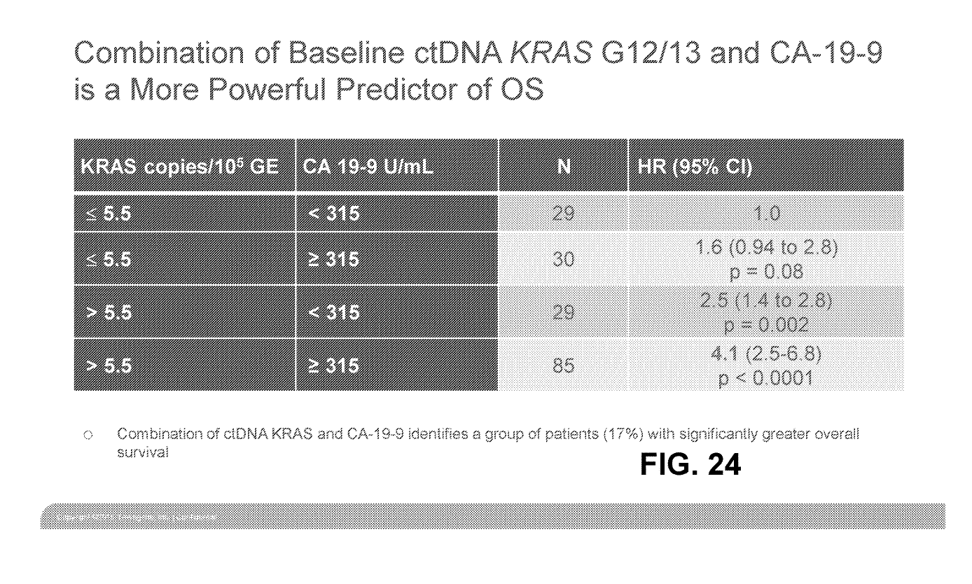

In some embodiments, the value of another molecular marker or a non-molecular marker at the various time points (e.g., baseline and two weeks) can increase the accuracy of the parameter being measured (e.g., overall survival). For example, combining KRAS determination with CA 19-9 determination at baseline is a more accurate predictor of overall survival than KRAS alone (FIG. 24).

These methods can be applied to predicting responsiveness to treatment with any cancer. In some embodiments, the cancer is adrenal cortical cancer, anal cancer, bile duct cancer, bladder cancer, bone cancer, brain or a nervous system cancer, breast cancer, cervical cancer, colon cancer, rectal cancer, colorectal cancer, endometrial cancer, esophageal cancer, Ewing family of tumor, eye cancer, gallbladder cancer, gastrointestinal carcinoid cancer, gastrointestinal stromal cancer, Hodgkin Disease, intestinal cancer, Kaposi sarcoma, kidney cancer, large intestine cancer, laryngeal cancer, hypopharyngeal cancer, laryngeal and hypopharyngeal cancer, leukemia, acute lymphocytic leukemia (ALL), acute myeloid leukemia (AML), chronic lymphocytic leukemia (CLL), chronic myeloid leukemia (CML), chronic myelomonocytic leukemia (CMML), non-HCL lymphoid malignancy (hairy cell variant), splenic marginal zone lymphoma (SMZL), splenic diffuse red pulp small B-cell lymphoma (SDRPSBCL), chronic lymphocytic leukemia (CLL), prolymphocytic leukemia, low grade lymphoma, systemic mastocytosis, splenic lymphoma/leukemia unclassifiable (SLLU), liver cancer, lung cancer, non-small cell lung cancer, small cell lung cancer, lung carcinoid tumor, lymphoma, lymphoma of the skin, malignant mesothelioma, multiple myeloma, nasal cavity cancer, paranasal sinus cancer, nasal cavity and paranasal sinus cancer, nasopharyngeal cancer, neuroblastoma, non-Hodgkin lymphoma, oral cavity cancer, oropharyngeal cancer, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma, adult soft tissue sarcoma, skin cancer, basal cell skin cancer, squamous cell skin cancer, basal and squamous cell skin cancer, melanoma, stomach cancer, small intestine cancer, testicular cancer, thymus cancer, thyroid cancer, uterine sarcoma, uterine cancer, vaginal cancer, vulvar cancer, Waldenstrom macroglobulinemia, or Wilms tumor. In various embodiments, the cancer is lung cancer, e.g., non-small cell lung cancer, colorectal cancer, or pancreatic cancer.

Any bodily fluid that would be expected to have nucleic acids can be utilized in these methods. Non-limiting examples of bodily fluids include, but are not limited to, peripheral blood, serum, plasma, urine, lymph fluid, amniotic fluid, and cerebrospinal fluid. In certain particular embodiments, such as those illustrated in the Examples, the bodily fluid is serum, plasma or urine. In various embodiments, cell-free DNA or RNA is determined. When the bodily fluid is urine, the nucleic acid may be transrenal cell-free DNA or RNA, e.g., as described in U.S. Pat. RE39920E1.

In any of the methods described herein, the mutation can be determined, or quantified, by any method known in the art. Nonlimiting examples include MALDI-TOF, HR-melting, di-deoxy-sequencing, single-molecule sequencing, use of probes, pyrosequencing, second generation high-throughput sequencing, SSCP, RFLP, dHPLC, CCM, or methods utilizing the polymerase chain reaction (PCR), e.g., digital PCR, quantitative-PCR, or allele-specific PCR (where the primer or probe is complementary to the variable gene sequence). In these methods, the mutation is quantified along with the wildtype sequence, to determine the percentage of mutated sequence (e.g., as genome equivalents, as in the example).

In many embodiments, the DNA is cell free DNA (“cfDNA”). In some embodiments, the amplified or detected DNA molecule is genomic DNA. In other embodiments, the amplified or detected molecule is a cDNA.

The skilled artisan can determine useful primers for PCR amplification of any mutant sequence for any of the methods described herein. In some embodiments, the PCR amplifies a sequence of less than about 50 nucleotides, e.g., as described in US Patent Application Publication US/2010/0068711. In other embodiments, the PCR is performed using a blocking oligonucleotide that suppresses amplification of a wildtype version of the gene, e.g., as described in U.S. Pat. No. 8,623,603 or PCT Patent Publication WO 2015/073163.

The treatment being assessed for responsiveness in these methods can be any cancer treatment, including surgery, chemotherapy, radiation therapy, hormone therapy, immunotherapy, or photodynamic therapy. Non-limiting examples of radiation therapy include external beam radiation therapy, such as with photons (gamma radiation), electrons, or protons; stereotactic radiation therapy, such as with a single high dose or multiple fractionated doses to a small target; brachytherapy; and systemic radioactive isotopes. Non-limiting examples of chemotherapy include cytotoxic drugs; antimetabolites, such as folate antagonists, purine antagonists, and pyrimidine antagonists; biological response modifiers, such as interferons; DNA damaging agents, such as bleomycin; DNA alkylating and cross-linking agents, such as nitrosourea and bendamustine; enzymatic activities, such as asparaginase; hormone antagonists, such as fulvestrant and tamoxifen; aromatase inhibitors; monoclonal antibodies; nucleic acids such as antisense agents, antibiotics such as mitomycin; platinum complexes such as cisplatin and carboplatin; proteasome inhibitors such as bortezomib; spindle poison such as taxanes or vincas or derivatives of either; topoisomerase I and II inhibitors, such as anthracyclines, camptothecins, and podophyllotoxins; tyrosine kinase inhibitors; anti-angiogenesis drugs; and signal transduction inhibitors. Non-limiting examples of hormonal therapy include hormone antagonist therapy, hormone ablation, bicalutamide, enzalutamide, tamoxifen, letrozole, abiraterone, prednisone, or other glucocorticosteroid. Non-limiting examples of immunotherapy include anti-cancer vaccines and modified lymphocytes.

In some embodiments, the treatment comprises targeted therapy. These embodiments are not narrowly limited to any particular targeted therapy. In some embodiments, the treatment is administration of a tyrosine kinase inhibitor, a serine/threonine kinase inhibitor, a compound targeting CD20, Her2/neu, the folate receptor, EGFR, PDGFR, KIT, VEGFR2 or a VEGF ligand. In certain more specific embodiments, the treatment comprises vinorelbine, gemcitabine, cisplatin, erlotinib, eocetaxel, bevacizumab, carboplatin, erlotinib, afatinib, rociletinib, AZD9291, crizotinib, ceritinib, alectinib, lapatinib, neratinib, or dabrafenib.

Through the prediction of responsiveness, the above method can be utilized as a tool in making treatment recommendations, specifically to stay with the treatment (e.g., if there is a significant spike in the mutation in the first week, indicating responsiveness to the treatment), or to change treatments (e.g., if there is no significant spike in the mutation in the first week, indicating lack of responsiveness). Thus, also provided herein is a method of determining treatment recommendations for a subject with cancer. The method comprises determining responsiveness of the subject by the above-described method, and (a) recommending continuation of the treatment if a spike in the quantity of the mutation was present within one week of starting the treatment, or (b) recommending a change of treatment if a spike in the quantity of the mutation was not present within one week of starting the treatment. In some embodiments, the significant increase is to greater than about 25, 50 or 100 copies of the mutation per 10⁵ genome equivalents (“GE”). In additional embodiments, the mutation associated with the cancer is in the EGFR gene, e.g., an EGFR activating mutation or EGFR T790M.

The prediction of treatment responsiveness or efficacy can also be utilized in treatment executions, specifically to stay with the treatment (e.g., if there is a significant spike in the mutation in the first week, indicating responsiveness to the treatment), or to change treatments (e.g., if there is no significant spike in the mutation in the first week, indicating lack of responsiveness). Thus, also provided herein is a method of treating a subject with cancer. The method comprises determining responsiveness of the subject by the above-described method, and (a) continuing the treatment if a spike in the quantity of the mutation was present within one week of starting the treatment, or (b) changing the treatment if a spike in the quantity of the mutation was not present within one week of starting the treatment. In some embodiments, the significant increase is to greater than about 25, 50 or 100 copies of the mutation per 10⁵ genome equivalents (“GE”). In additional embodiments, the mutation associated with the cancer is in the EGFR gene, e.g., an EGFR activating mutation or EGFR T790M, or a KRAS gene, e.g., KRAS G12D, G12S, or G13D.

These methods can also be utilized to determine the progression and effectiveness of treatment of transplant rejection or other diseases such as chronic viral (e.g., HIV, HCV, herpes), bacterial (e.g., tuberculosis) or other pathogen infections (e.g., parasitic infections such as by Enterobius vermicularis, Giardia lamblia, Ancylostoma duodenale, Necator americanus, and Entamoeba histolytica. The methods for these diseases are analogous to those described above for cancer. Samples of a bodily fluid such as urine or blood are taken periodically before, during and/or after treatment and cell-free nucleic acids associated with the disease (e.g., HIV, M. tuberculosis, or parasitic nucleic acids) or transplant (e.g., nucleic acids characteristic of the transplated tissue) are quantified, and the effectiveness of treatment is evaluated based on whether the nucleic acids are present and/or have changed in quantity.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3rd edition), Cold Spring Harbor Press, Cold Spring Harbor, N.Y. (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18th edition (1990). These texts can, of course, also be referred to in making or using an aspect of the disclosure.

Preferred embodiments are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Example 1. Urine Testing for EGFR T790M and Prognosis in Lung Cancer

Twenty two non-small cell lung carcinoma patients being treated with erlotinib or afatinib (+/−radiation) were monitored longitudinally for early acquisition of the EGFR T790M mutation (“T790M”) using urinary cell-free circulatory tumor DNA (“ctDNA”) by the methods described in PCT patent application PCT/US14/61435. Concordance between urine and tissue was assessed. Of the 22 patients, 13 were tested for T790M by CLIA tissue biopsy (10 were positive), and 4 patients were tested for T790M by plasma (3 were positive).

Longitudinal specimens were collected for up to 4 months prior to progression on anti-EGFR therapy, collection frequency 3-6 weeks. The 10 patients that were positive for T790M by tissue biopsy received treatment with an exploratory 3rd generation anti-EGFR T790M tyrosine kinase inhibitor (“TKI”). To monitor early kinetics of drug response, urine specimens were collected from 9 patients prior to treatment, on treatment daily for 1 week, then weekly for 3 weeks, then monthly.

### Results

### Sensitivity of the Tissue, Plasma and Urine Tests

The EGFR T790M mutation was detected as early as 3 months prior to radiological detection of progression on first line anti-EGFR TKI treatment, showing the effectiveness of mutation detection for determining cancer presence.

The EGFR T790M mutation was detected in 15 of 22 (68%) patients receiving anti-EGFR treatment (detection at any time points).

Ten of 10 patients who were treated with anti-T790M TKI (tissue T790M-positive), were found to be positive for T790M in urine at any time point, showing the effectiveness of urine testing in detecting T790M.

In 8 of 9 patients with pre-treatment urine specimens available, the T790M mutation was detected prior to anti-T790M treatment. In the ninth patient, the T790M mutation was undetectable at baseline but detected while on anti-T790M treatment. In 1 of 10 patients who did not have a pre-treatment urine specimens, the T790M mutation was detected in urine while on anti-T790M treatment. This further shows the effectiveness and sensitivity of urine testing for detecting cancer biomarkers.

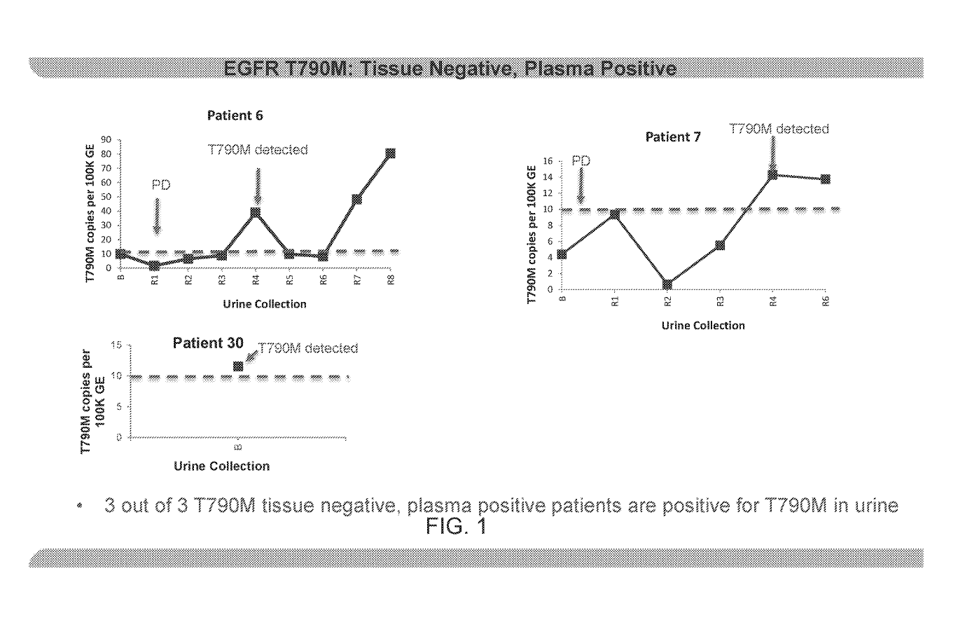

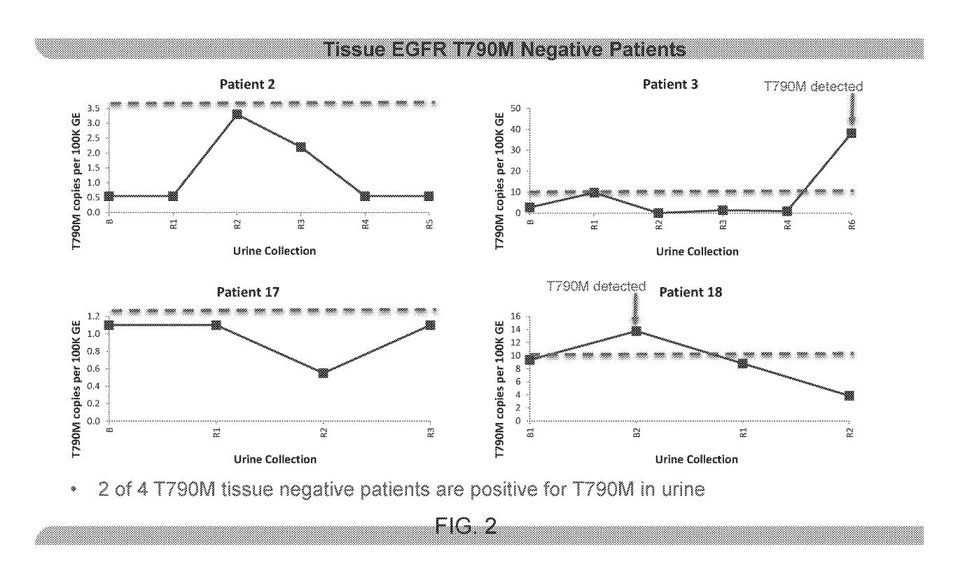

As shown in FIG. 1, three out of three T790M tissue negative, plasma positive patients were positive for T790M in urine, indicating a higher sensitivity of the urine tests over the tissue tests. This is further supported by FIG. 2, showing testing on four T790M tissue negative patents with the urine tests, where two of the four tissue negative patients were positive for T790M in urine.

Of 15 patients positive for T790M by urine, 4 patients were tested by plasma. The T790M mutation was detected in 3 of 4 urine-positive patients. This indicates that the urine test is at least as sensitive or more sensitive for T790M than the plasma test.

Dynamics of T790M after Treatment

As shown in FIGS. 3-5, when T790M positive patients were treated with an exploratory anti-T790M drug, a decrease in ctDNA T790M load was observed as early as 4 hours or 1 day on treatment. The initial decrease in urinary T790M was followed by a spike in T790M during the first week of therapy. The size of the spike during week 1 correlated with clinical response: patients with T790M spike above 25 copies/10⁵ genome equivalents (“GE”) had a partial response to the treatment, while patients with a spike below 25 copies/10⁵ GE or no spike during week 1 had stable disease.

### Example 2. Predicting Radiographic Response and Early Assessment of Targeted Therapy by Monitoring EGFR Mutations in Urine

Lung cancer patients with two EGFR mutations were monitored by quantifying the two mutations in urine samples taken from the patients before commencing a seven day EGFR-targeted tyrosine kinase inhibitor therapy, and daily during the therapy, then at various subsequent times. The results from Patient 1, monitored for EGFR mutations T790M and Exon 19del are shown in FIG. 6 and Table 1. FIG. 6A shows 13 weeks of monitoring, with computed tomographic (CT) imaging results; FIG. 6B shows the first week of daily measurements for Patient 1. Within two days of the start of treatment, a spike in both mutations of greater than 25-100 copies per 100,000 genome equivalents (GE), but not greater than about 1,000 copies per 100,000 GE, followed by undetectable amounts of the mutations within two weeks of commencement of treatment predicted the partial response shown by the slow decrease in tumor size in the 6 week and 12 week CT scan.

TABLE 1
Quantities of mutant EGFR during treatment

| | T790M | Exon 19del |
|---|---|---|
| | Copies/100K geq | Copies/100K geq |
| Time on Drug | (95% CI) | (95% CI) |
| Day 0 | 24 (19-38) | 167 (125-267) |
| Day 1 (4 hrs) | <LOD | 8 (6-13) |
| Day 1 | 221 (168-361) | 87 (65-139) |
| Day 2 | 34 (28-55) | 117 (88-187) |
| Day 3 | 48 (39-78) | 36 (27-58) |
| Day 4 | <LOD | <LOD |
| Day 5 | 15 (13-25) | <LOD |
| Day 6 | <LOD | 19 (14-30) |
| Day 7 | <LOD | <LOD |
| Week 2 | <LOD | <LOD |
| Week 3 | <LOD | <LOD |
| Week 4 | <LOD | <LOD |
| Week 6 | <LOD | <LOD |
| Week 12 | <LOD | <LOD |

Limit of detection (LOD):
T790 = 2 copies (12 copies/100K Genome Equivalents [GE])
Exon 19del = 1 copy (6 copies/100k GE)

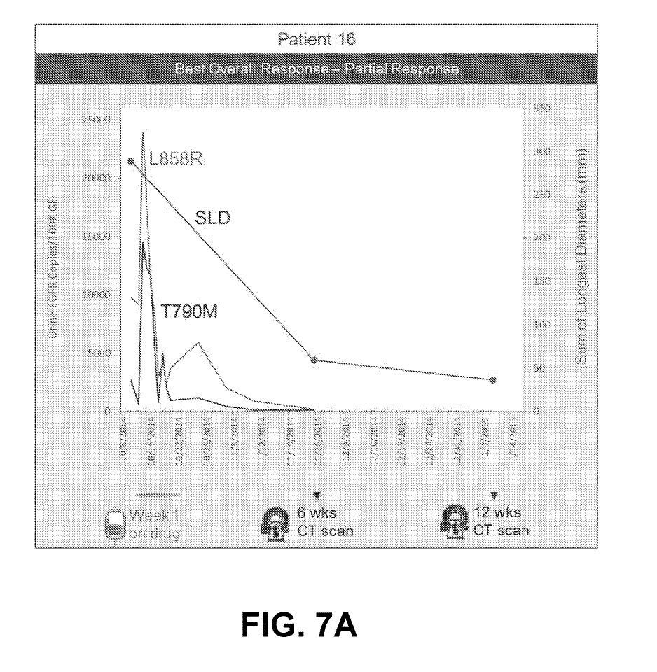

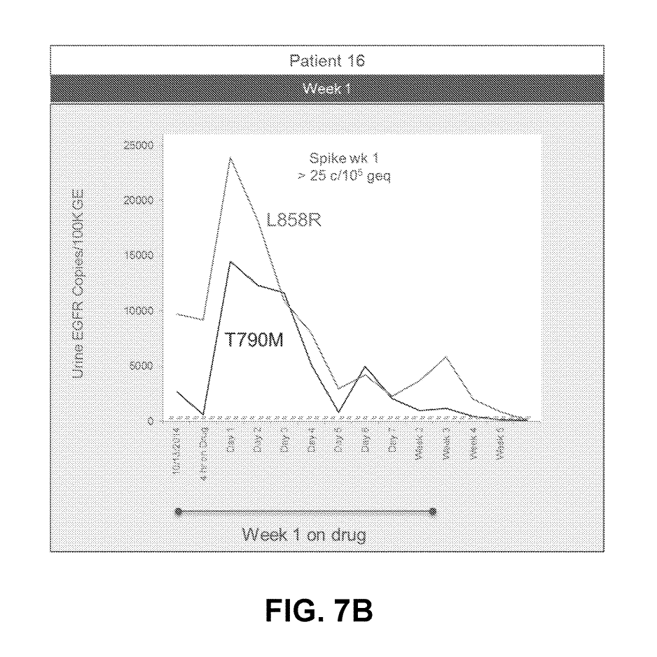

Patient 16, monitored for EGFR mutations T790M and L858R, exhibited a spike at Day 1 of greater than 10000 copies per 100,000 GE, then a decrease to below the limit of detection by week 6, foretold a strong partial response (FIGS. 7A and 7B).

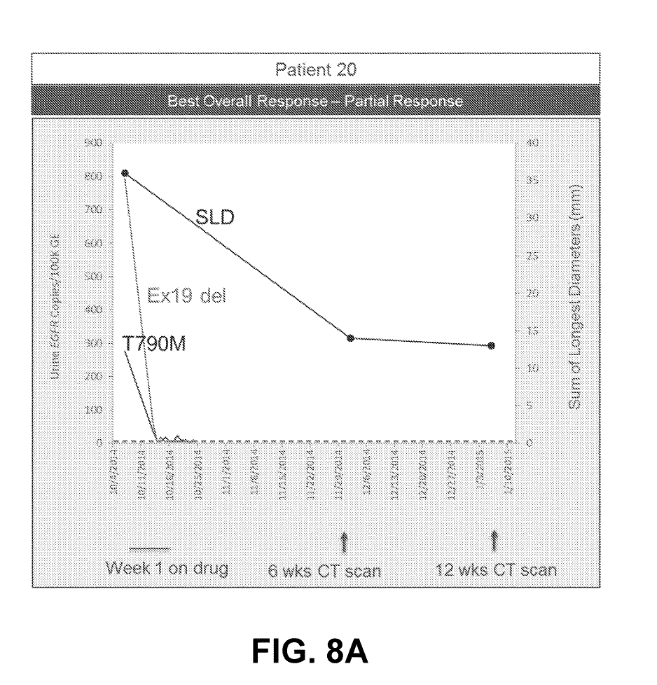

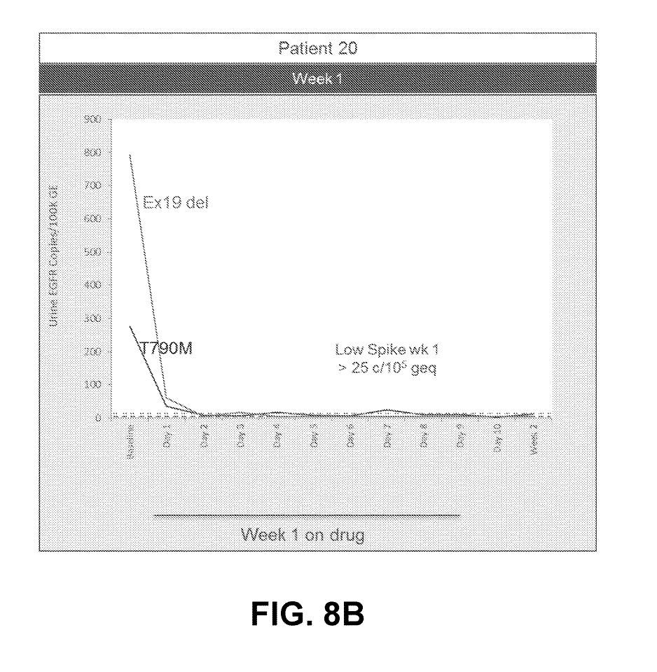

The results from Patient 20, monitored for EGFR mutations T790M and Exon 19del are shown in FIGS. 8A and 8B. A sharp decrease in to below the limit of detection within two weeks of the start of treatment predicts the partial response in lesion diameter.

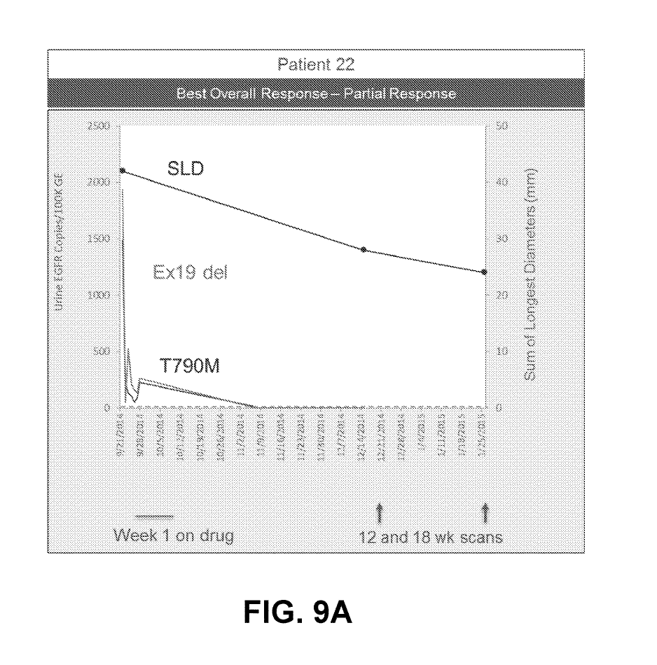

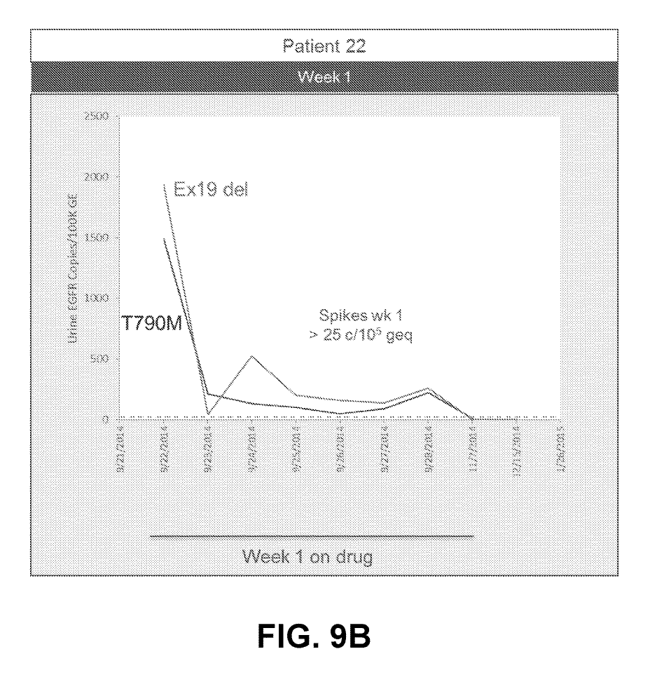

Patient 22, monitored for EGFR mutations T790M and Exon 19del, exhibited, within a week of the start of treatment, a spike in both mutations of greater than 25-100 copies per 100,000 GE, but not greater than about 1,000 copies per 100,000 GE, predicting the partial response (FIGS. 9A and 9B).

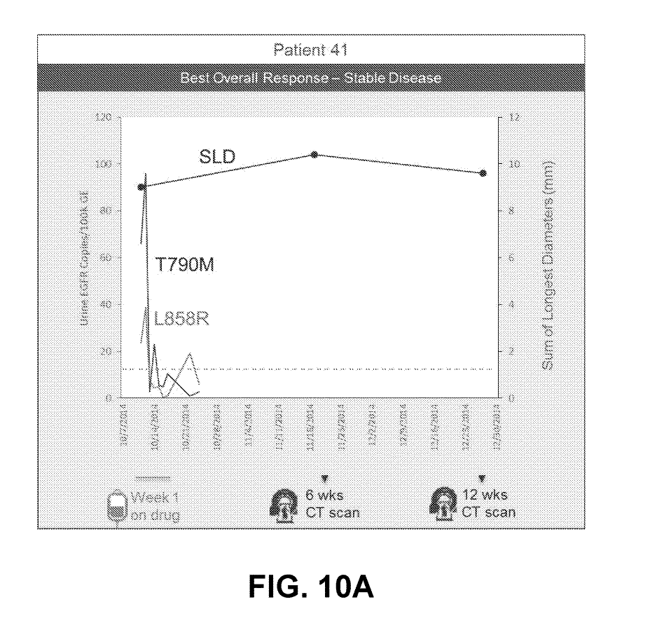

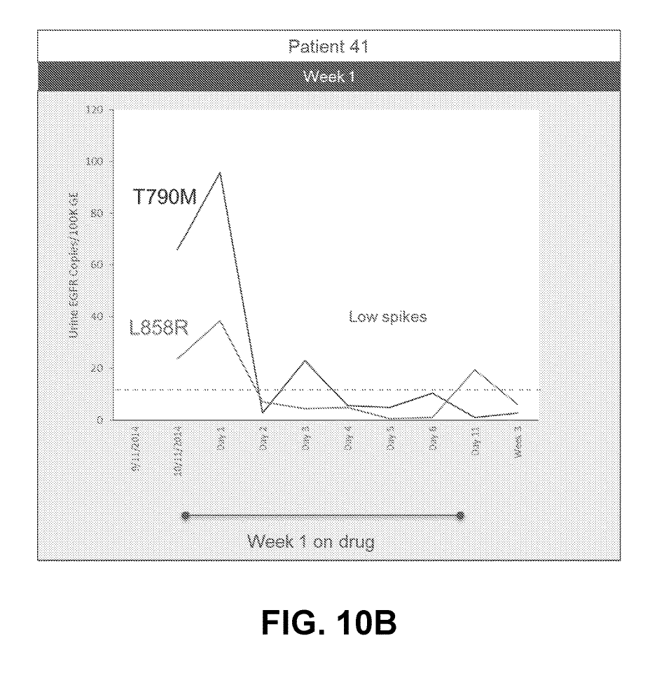

Patient 41, monitored for EGFR mutations T790M and L858R, exhibited small spikes for both mutations at Day 1, with the L858R spike less than 50 copies per 100,000 GE. This predicted stable disease (FIGS. 10A and 10B).

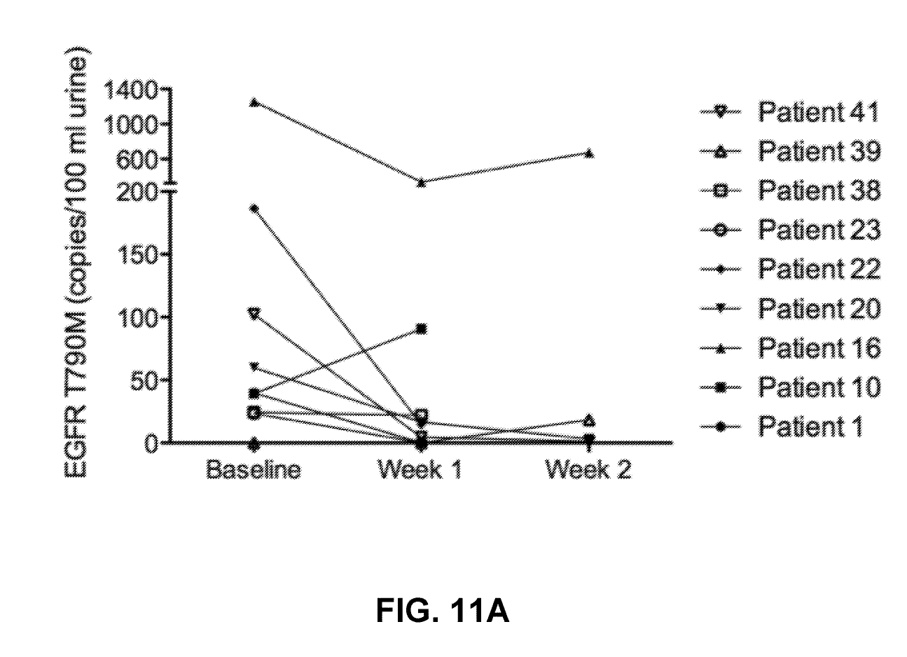

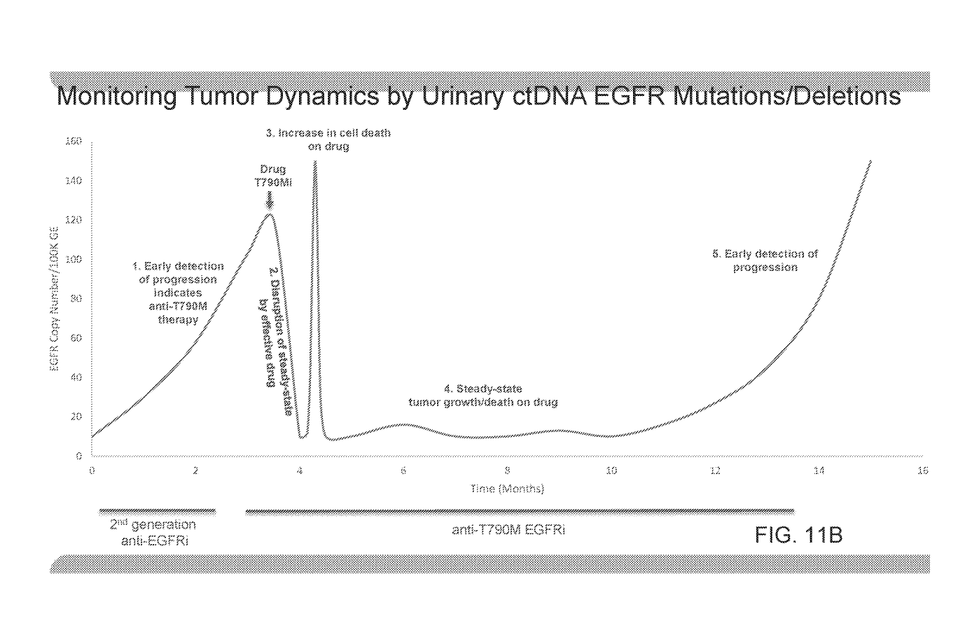

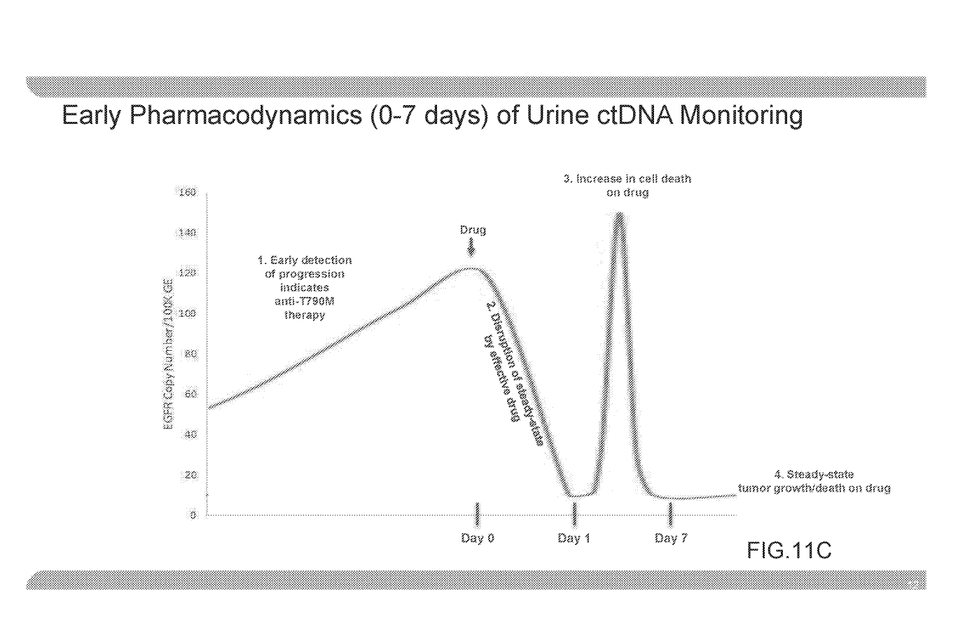

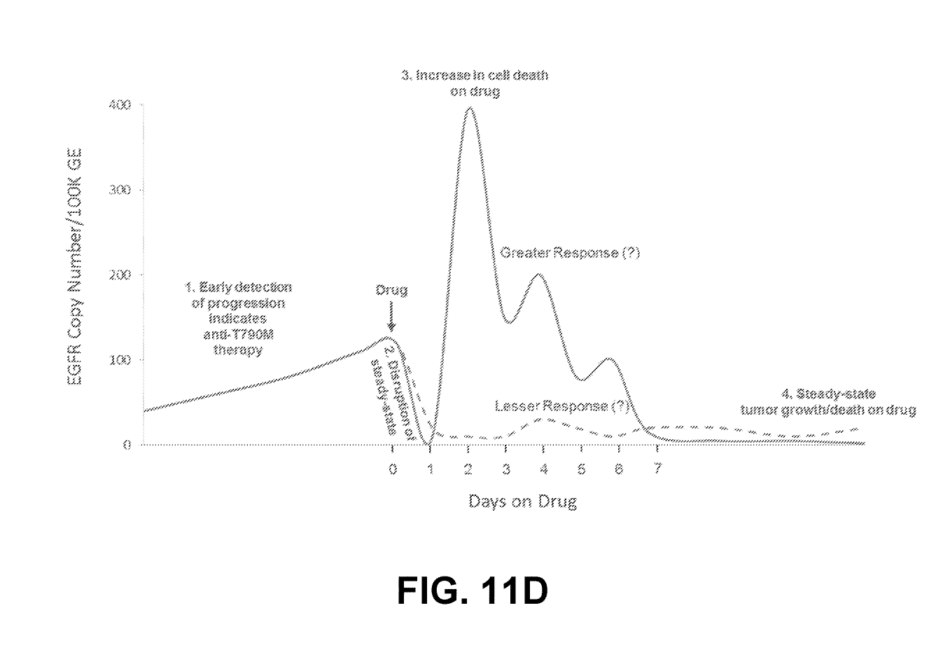

A summary of the reduction in urinary ctDNA EGFR mutational load after 1 or 2 weeks on anti-EGFR T790M treatment is shown in FIG. 11A. Those observations and the associated responses show that a large spike in urine-detectable mutations within a week of the start of treatment, i.e., greater than about 1000 copies per 100,000 GE, indicates a greater response than a spike of between about 100 and 1000 copies per 100,000 GE, while a spike of less than about 25-100 copies per 100,000 GE indicates a poor response or stable disease. This responsive outcome is illustrated in the model provided as FIG. 11B; FIG. 11C shows mutant levels in the first week of this typical responsive outcome. Without being bound by any particular mechanism for the correlation between the size of the spike of mutant DNA in urine within a week of the start of treatment and responsiveness to treatment, the model indicates that the spike results from an increase in apoptosis of cancer cells from the drug. This hypothesis is consistent with the observed larger spike with greater responsiveness, since a greater responsiveness to the drug would logically lead to more cell death and a larger spike. It is surprising that the spike occurs so soon after the start of therapy, often within one day.

A model that includes typical non-responsive outcomes with responsive outcomes is shown in FIG. 11D, where a poorer response or no response exhibits a lower or nonexistent spike in mutant levels in the first week when compared to a responsive treatment.

While these models are exemplified here as quantifying the mutant EGFR T790M, which is a “resistance mutation” that can arise after treatment of the original cancer that has a different cancer mutation, these models also hold true with a responsive or non-responsive treatment with of a cancer having the original mutation.

These models, exemplifying a treatment to a resistance mutation, shows an increase in the resistance mutation before treatment (Day 0). This increase can be detected through monitoring for minimal residual disease (MRD) before the relapse can be detected radiologically.

### Example 3. Monitoring Daily Dynamics of Early Tumor Response to Targeted Therapy by Detecting Circulating Tumor DNA in Urine

### Example Abstract

Non-invasive drug response biomarkers for early assessment of tumor response can enable adaptive therapeutic decision-making and proof-of-concept studies for investigational drugs. Circulating tumor DNA (ctDNA) is released into the blood by tumor cell turnover and subsequently excreted in urine. We tested the hypothesis that dynamic changes in EGFR activating and resistance (T790M) mutation levels detected in urine could inform tumor response within days of therapy for advanced non-small cell lung cancer (NSCLC) patients receiving osimertinib (AZD9291). Eight of nine NSCLC patients had detectable T790M-mutant DNA fragments in pre-treatment baseline samples. Daily monitoring of mutations indicated a pattern of overall decrease in fragment numbers between baselines to day 7 with intermittent peaks throughout week 1 preceding radiographic response at 6-12 weeks. Findings suggest osimertinib-induced tumor apoptosis within days of initial dosing. Daily urine sampling of ctDNA could enable early assessment of patient response and proof-of-concept studies for drug development.

### Introduction

Non-invasive drug response biomarkers for early assessment of tumor response with correlation to patient outcome could greatly impact therapeutic decision-making for the multiple targeted therapy options currently available for cancer treatment. Furthermore, non-invasive pharmacodynamic biomarkers are needed to determine early tumor response by experimental targeted therapies for demonstrating proof-of-concept (e.g., drug-induced apoptosis) (Gainor et al., 2014). Morphological or functional assessment of tumor burden using computed tomography (CT), magnetic resonance imaging (MRI) or positron emission tomography (PET) remains the standard of care for response assessment. However, imaging lacks fundamental information regarding the tumor DNA mutation status and therefore intrinsic tumor biology. Furthermore, conventional imaging modalities can be subject to confounding variables that mimic tumor progression or response depending on the tumor type (i.e., pseudo-progression or pseudo-response) (Kurzrock et al., 2013). Strategies that characterize molecular analysis of tumor DNA mutation status through repeat tissue biopsies for therapeutic decision-making or proof-of-concept evaluation of investigational drugs are employed in lung cancer management, but are increasingly becoming a less viable option given the invasiveness of the procedure, potential complications associated with the biopsy procedure, practical concerns around the scheduling and frequency of testing, and potential lack of assessment of intra- and inter-tumor heterogeneity (Diaz and Bardelli, 2014).

Circulating tumor DNA (ctDNA) is released into the blood from tumor cells with greater amounts present as tumor volume and subsequent cellular turnover increase (Diaz and Bardelli, 2014; Jahr et al., 2001; Schwarzenbach, 2011). ctDNA is highly degraded (˜180-200 bp) with classic apoptotic DNA size laddering and is most likely derived from apoptotic turnover of tumor cells; the proportion of ctDNA to total cell-free wild-type (WT) DNA present in blood varies widely from very rare (0.01%) to highly prevalent (>90%) and is patient and tumor-burden dependent (Diaz and Bardelli, 2014; Jahr et al., 2001; Schwarzenbach, 2011). ctDNA biomarkers in blood can be concordant with patient-matched tissue biopsies, can identify intra- and inter-tumor heterogeneity, and can correlate with responsiveness to therapy (Diaz et al., 2012; Haber and Belculescu, 2014; Leary et al., 2012; Piotrowska et al., 2015; Bettegowda et al., 2014; Janku et al., 2014; Karachaliou et al., 2015; Newman et al., 2014; Siravegna et al., 2015; Thress et al., 2015). ctDNA present in blood is excreted into urine, and patient-matched tissue, plasma and urine studies indicate concordance of DNA mutation status across all three biopsy specimens (Janku et al., 2014; Hyman et al., 2015; Melkonyan et al., 2008; Su et al., 2004). ctDNA detection and quantitation by urine sampling provides a non-invasive source of ctDNA from cancer patients that readily enables daily urine collection. This sampling flexibility was leveraged to determine whether detection and quantitation of ctDNA biomarkers in urine could assess early tumor response within days of a patient receiving targeted therapy.

We tested this hypothesis by monitoring the daily tumor dynamics of EGFR-activating mutations (L858R, exon 19 deletions) and resistant mutation T790M in urine from patients with metastatic non-small cell lung cancer (NSCLC) receiving osimertinib. Osimertinib is highly active against EGFR T790M-bearing NSCLC with a complete and partial response (CR and PR) rate of 61% and a clinical benefit rate of disease control of 95% (CR, PR, stable disease (SD) (Janne et al., 2015). Osimertinib was recently approved by the US Food and Drug Administration for the treatment of patients with metastatic EGFR T790M mutation-positive NSCLC (http://www.fda.gov/drugs/informationondrugs/approveddrugs/ucm472565.htm).

### Methods

Patients.

Ten patients with NSCLC undergoing treatment with erlotinib were enrolled in the study. Tissue biopsies were performed as part of routine clinical care, with the site of biopsy based on radiographic and/or clinical assessment of disease involvement. Between 60-120 mL of urine were collected at each time point. All urine samples were de-identified for the staff performing ctDNA testing, and operators performing plasma and urine cfDNA analyses were blinded to the tissue genotype and clinical characteristics of all patients. For early response monitoring, osimetrinib was first administered on day 0 (baseline) and then continued until progression. Daily first morning urine voids were collected before drug administration. The study was performed and consent obtained in accordance with UCSD IRB guidelines.

Analysis of Tissue Biopsies.

Molecular analysis of formalin-fixed paraffin-embedded (FFPE) tumor tissue biopsies was performed within 28 days of radiologic progression on first-line anti-EGFR TKI at a central lab (LabCorp) using the Cobas® EGFR Mutation Test (Roche Molecular Systems).

Radiographic Assessments.

The overall response rate was assessed according to RECIST 1.1 by both the investigator and an independent central review. Patients were assessed at baseline, and every 6 weeks from the time of first dose; participants will be followed by CT/MRI scans for RECIST 1.1 until the date of progression.

ctDNA EGFR Mutational Analysis

Urinary ctDNA Extraction.

Urine was collected in 110 mL collection vessels; proprietary preservative was added immediately after urine collection. Urine was concentrated using a Vivacell 100 (Sartorius Corp, Bohemia N.Y.) and then processed using a two-step DNA extraction method. Briefly, concentrated urine was mixed with 700 uL of Q-sepharose Fast Flow quaternary ammonium resin (GE Healthcare, Pittsburgh, Pa.) and 20 mL binding buffer (100 mM Tris, 50 mM EDTA, 0.02% Tween, pH 8). Following incubation at room temperature for 1 hour, tubes were spun to collect sepharose and bound DNA. The pellet was then resuspended in a buffer containing guanidinium hydrochloride and isopropanol, and the eluted DNA was collected as a flow-through using polypropylene chromatography columns (BioRad Laboratories, Irvine, Calif.). The eluate was further purified using QiaQuick columns (Qiagen, Germany).

Quantitative ctDNA Analysis.

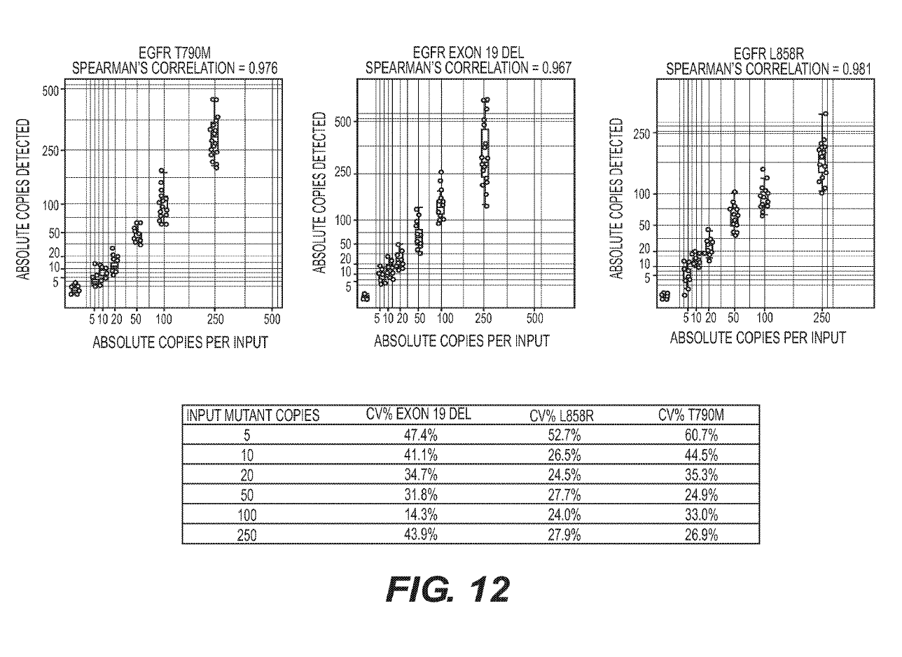

Extracted DNA was quantitated using a droplet digital PCR (ddPCR) assay that amplifies a single copy RNaseP reference gene (QX200 ddPCR system, Bio-Rad, CA), as described previously (Janku et al., 2014). Quantitative analysis of EGFR activating mutations and T790M resistance mutation was performed using mutation enrichment PCR coupled with next-generation sequencing detection (MiSeq, Illumina Inc., CA). Mutation enrichment was accomplished via a short amplicon, kinetically driven enrichment PCR that selectively amplifies mutant fragments while suppressing amplification of the wild-type (WT) sequence using blocker oligonucleotide. Following enrichment PCR, custom DNA libraries were constructed and indexed using Access Array System for Illumina Sequencing Systems (Fluidigm Corp, San Francisco, Calif.). The indexed libraries were pooled, diluted to equimolar amounts with buffer and the PhiX Control library, and sequenced to 200,000× coverage on an Illumina MiSeq platform using 150-V3 sequencing kits (Illumina, Inc. CA). Primary image analysis, secondary base-calling and data quality assessment were performed on the MiSeq instrument using RTA v1.18.54, and MiSeq Reporter v2.6.2.3 software (Illumina Inc., CA). Analysis output files (FASTQ) from the run were processed using custom sequencing reads counting and variant calling algorithm to tally the sums of total target gene reads, wild-type (WT) or mutant EGFR reads that passed sequence quality criteria (qscore ≧20). Custom quantification algorithm was developed to accurately determine the absolute number of mutant DNA molecules in the source ctDNA sample. To that end, each single multiplexed MiSeq NGS run contained, in addition to clinical samples and controls, 12 standard curve samples (3 replicates with known mutant input copies at 4 levels). Mutant reads in a test sample were converted to absolute mutant copy number in the original sample by interpolation to the standard curve. Testing of analytical performance of the EGFR mutation detection assays demonstrated that absolute measurements by mutation enrichment NGS across three EGFR assays corresponded to 107%±40.2% of input mutant copies, with mean Coefficient of Variation Percent (CV %) of 34.5% across 5-250 input mutant copy range, indicating that the absolute detection by enrichment PCR-NGS is remarkably efficient (FIG. 12).

Clinical EGFR Mutation Detection Cut-Offs.

Clinical EGFR mutation detection cut-offs were determined by analyzing 200 urine DNA samples obtained from unique healthy volunteers and metastatic patients with wild-type EGFR status as determined by CLIA local laboratory testing of tumor tissue FFPEs. Mutation-specific cut-offs were set to the median plus three standard deviations of the mutant EGFR copy counts in the urine samples from EGFR mutation-negative population. Detection cut-offs were standardized to 100,000 WT genome equivalents (GEQ) yielding adjusted clinical detection cut-offs of 5.5, 5.5 and 12.6 for exon 19 deletions, L858R and T790M, respectively.

Statistical Analysis.

Analysis of trends observed in urine ctDNA EGFR signal upon patient treatment with anti-EGFR tyrosine kinase inhibitor was assessed using Wilcoxon's paired two-sample test with a one-sided p-value. P values less than 0.05 were considered statistically significant. Correlation between input and output absolute EGFR mutant copies in the analytical spike-in experiments was examined using Spearman's correlation, which allows to account for the non-linearity of variables. Analytical variability of the assays was examined using the Coefficient of Variation Percent (CV %), calculated as the ratio of the standard deviation to the mean of the absolute EGFR copies detected within each absolute copy per input level and is reported as a percentage. All statistical analyses were carried out using R v3.2.3 computer software.

### Results

Detection of ctDNA Mutant EGFR DNA Fragments in Urine

To overcome the inherent technical challenges of detecting degraded ctDNA having rare prevalence within cell-free WT DNA, assays for the detection of EGFR-activating mutations (exon 19 deletions, L858R) and resistance mutation T790M were developed to generate short amplicon lengths of 33 bp, 46 bp and 44 bp, respectively. Subsequent polymerase chain reaction (PCR) amplification using wild-type blockers was done to enrich for mutant ctDNA, and quantitation of mutant sequences was completed by next generation sequencing. Using this approach, the analytical Lower Limit of Detection (LLoD) of the exon 19 deletions, L858R and T790M assays was 1, 1 and 2 copies respectively in the background of approximately 18,180 WT genome equivalents or a mutant fraction range of 0.006-0.01%. Copies reported herein are standardized to 100,000 WT genome equivalents (geq) yielding an adjusted lower LLoD's of 5.5, 5.5 and 11 for exon 19 deletions, L858R and T790M, respectively. Concurrent standard curves were assayed with patient samples for accurate determination of the absolute number of mutant DNA molecules in each urine sample.

Ten patients with locally advanced or metastatic NSCLC and radiologically documented progression from treatment with an EGFR TKI were enrolled in this prospective study; nine had available serial urine samples including a sample at baseline. All nine patients had a positive tissue biopsy for both the EGFR-activating mutation (exon 19 deletion or L858R) and the resistant mutation T790M (Table 2). Prospectively collected urine samples were processed without knowledge of subsequent patient response. Detectable T790M mutant DNA fragments were observed in baseline urine samples in 8 out of 9 patients (median=40 copies; range 18 to 2,684) with concordant EGFR-activating mutation (L858R, exon 19 deletions) DNA fragments in 7 of 8 patients (median=34 copies; range 10 to 9,745); one patient had a tissue biopsy EGFR exon 21 L861Q mutation that was not assayed in urine (Table 2, Patient 10). Overall, the percent of mutant EGFR fragments versus WT DNA ranged over 100-fold from 0.033% to 12.4% in urine DNA.

TABLE 2
Detection of mutant EGFR in urine of patients with NSCLC
who had relapsed on first-line, anti-GFR therapy.

| | | | # of EGFR-activating |
|---|---|---|---|
| | | | mutation (L858R |
| | | # of EGFR | and/or exon 19 |
| | | T790M | deletions) |
| | | molecules | molecules |
| Patient ID | Tissue Mutation | per 10⁵ geq¹ | per 10⁵ geq |
| 1 | T790M, exon 19 del | 34 | 167 |
| 10 | T790M, L861Q | 45 | N/A² |
| 16 | T790M, L858R | 2684 | 9745 |
| 20 | T790M, exon 19 del | 276 | 793 |
| 22 | T790M, exon 19 del | 2111 | 1932 |
| 23 | T790M, exon 19 del, | 34 | 43 |
| | L858R | | |
| 38 | T790M, exon 19 del | 18 | 10 |
| 39 | T790M, exon 19 del | <LLoD³ | <LLoD³ |
| 41 | T790M, L858R | 94 | 24 |

¹Abbreviations: EGFR = epidermal growth factor receptor; geq, genome equivalents;
²N/A: L861Q mutation was not tested;
³LLoD: number of mutant molecules below Lower Limit of Detection: NSCLC = non-small cell lung cancer

### Monitoring Early Tumor Response to Osimertinib in Urine

To quantitate and trend the dynamics of the EGFR mutational load in urine of patients treated with osimertinib, ctDNA was assessed at baseline, followed by collection of daily samples for seven days and then weekly samples. All 8 patients with detectable T790M baselines achieved clinical benefit when treated with osimertinib, as evidenced by the radiographic assessment at 6 and 12 weeks after therapy: seven patients had PR after the treatment, and one patient (patient 41) had SD for six months by sum of the longest diameters of lesions.

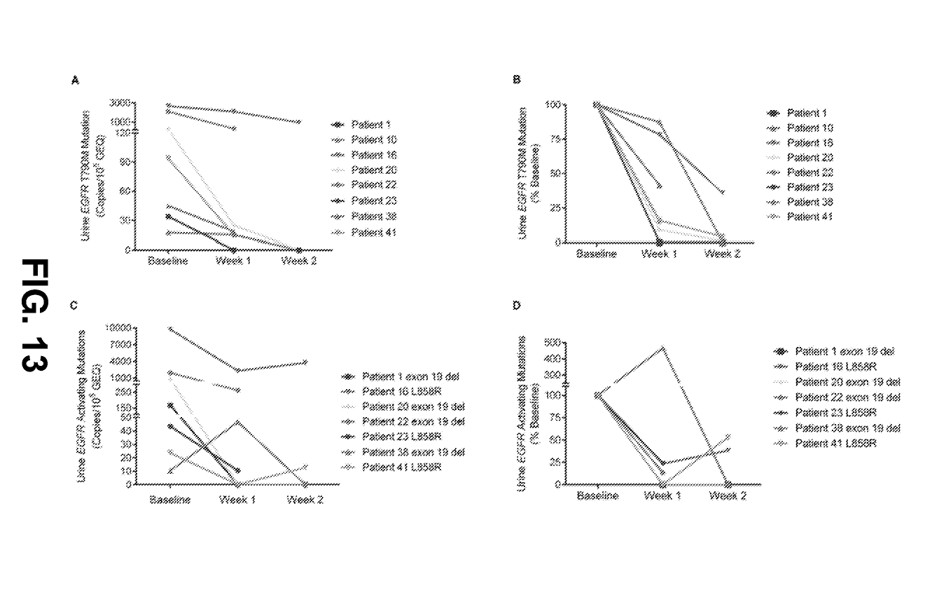

Overall, for all eight patients, there was a large decrease in both the number and percent of copies detected at week 1 and 2 compared to baseline for the resistance EGFR mutation T790M and EGFR-activating mutations L858R and exon 19 deletions (FIG. 13). A significant decrease was observed for the relative change from T790M baseline to week 1 (median 66.5% decrease, p=0.014) and week 2 (median 100% decrease, p=0.045). A similar decreasing trend was observed for EGFR-activating mutations L858R and exon 19 deletions with a median 86% and 81% decrease in signal at weeks 1 and 2, respectively (FIG. 13B,D). Daily monitoring for T790M ctDNA indicated a consistent pattern of a rapid overall decrease in the numbers of copies with patient-dependent intermittent peaks distributed over the first week of therapy (FIG. 14). In some patients, the spike in mutant ctDNA followed by a precipitous decrease was observed by day 4. Similar matching kinetics of early response were observed for the corresponding EGFR-activating mutations L858R and exon 19 deletions with overall numbers of copies mostly higher than for T790M. Following these early temporal peaks, low steady-state levels of ctDNA EGFR appeared to be established after 1 to 2 weeks on treatment (FIG. 14) with subsequent levels continuing to be at low steady-state in those patients in which subsequent urine samples were available. Both the temporal peaks and the subsequent rapid loss of mutant EGFR ctDNA signals in urine after the first 1 to 2 weeks of treatment were associated with and preceded the detection of radiographic response, as measured 6 and 12 weeks following the initiation of therapy.

### Discussion

Daily monitoring of ctDNA by non-invasive urine sampling of patients receiving targeted therapy readily enables temporal and quantitative dissection of early tumor response. Both the overall decrease and patient-dependent intermittent peaks of levels for EGFR-activating mutations L858R and exon 19 deletions and resistant mutation T790M during the first week indicate that osimertinib induces tumor cell apoptosis within days of drug administration to patients with NSCLC. Pharmacokinetic data for osimertinib is consistent with this observation, with an indicated median time to maximum levels in blood of 6 hours (Cₘₐₓ) and a mean half-life of 55 hours. Additionally, previous preclinical studies of osimertinib in tumor xenograft mouse models demonstrated a strong inhibition of both phospho-EGFR and downstream signaling pathways within six hours and significant tumor shrinkage at day 7 from dose initiation (Cross et al., 2014).

ctDNA monitoring in urine has potential utility to act as an early evidentiary pharmacodynamic biomarker for proof-of-concept studies of targeted therapies in development. Currently, in 2015, the number of oncology investigational drugs in the US is quite large, with 771 drugs or vaccines in development (98 in lung cancer alone) and 3,137 clinical trials being conducted (Buffery, 2015). Specifically, this approach could be used to determine whether an investigational drug is inducing apoptosis of the targeted tumor cells (i.e., drug target inhibition) by quantitating daily changes in urine ctDNA levels of the targeted tumor DNA mutation(s). In addition, for chemotherapies and immunotherapies that do not target a specific tumor genomic alteration, tumor response could be determined by quantitating levels of tumor DNA mutation(s) prevalent for the tumor type under investigation.

In this study, we observed, within one to two weeks of therapy, and in some patients as early as day 4, a large decrease relative to baseline for both the EGFR-activating mutations L858R and exon 19 deletions and the resistance mutation T790M, with intermittent peaks prevalent in all patients. This first week pattern is associated with subsequent clinical benefit for all eight patients (7 PR, 1 SD) by radiographic assessment at 6 and 12 weeks after therapy initiation. Our findings are consistent with previously reported studies in plasma demonstrating an overall decrease in ctDNA levels for patients responding to targeted therapy weeks to months after initiation of therapy (Siravegna et al., 2015; Marchetti et al., 2015; Dawson et al., 2013). Urine sampling enables the ability to probe tumor response within an immediate time window of the first week of therapy, with daily non-invasive assessment of drug-induced tumor cell apoptosis. Our findings further demonstrate that an earlier evaluation of patient response may be obtained for targeted therapy using urine, with an informative, predictive decrease of mutational load within 1 to 2 weeks of therapy. This paves the way for a practical opportunity to intervene earlier with combinatorial strategies that anticipate resistance.

A desirable fundament in cancer therapeutic decision-making is to have the ability to make an early assessment of patient responsiveness to therapy, and facilitate a new paradigm in individualized patient care. As the number of targeted therapies and combinations thereof increase, there is a strong clinical need for non-invasive tumor genomic testing with flexibility of testing tailored to the clinical context for an individual patient. Radiographic assessment of tumor burden after initial drug administration may hamper this objective in optimal patient care. Faster assessment of patient response can aid in navigating adaptive therapy strategies to reduce drug toxicity, identify resistance to therapy and enable consideration of other therapies. Here, we have demonstrated that non-invasive early assessment of tumor response by urine ctDNA monitoring within the first weeks of therapy has the potential to predict likelihood of patient response to targeted therapies.

### Example 4. Urine and Plasma Testing of KRAS Mutations in Colorectal Cancer

A quantitative circulating tumor DNA (“ctDNA”) assay using a massively parallel deep sequencing approach was developed to monitor ctDNA KRAS Exon 2 mutational load in plasma and urine. This ultrasensitive assay detects a single copy mutant KRAS DNA in a background of 18,181 wild-type genomic equivalents (0.0055% sensitivity).

In a blinded study of colorectal cancer (“CRC”) patients with known KRAS mutational status in tumor tissue, a correct KRAS mutation was identified in 95% of archival plasma and 92% of archival urine specimens.

A clear correlation and compatible fold change was demonstrated for the first time between the dynamics of plasma and urinary ctDNA KRAS changes on treatment (surgery and adjuvant). In all 5 patients with curative intent surgery (FIG. 15 provides a representative example), ctDNA KRAS levels were undetectable in urine or plasma after surgery. In contrast, in 10 of 11 patients with incomplete, palliative surgery (FIG. 16 provides four examples), the ctDNA KRAS signal remained detectable or increased after surgery.

This demonstrates clinical applicability of assessing the minimal residual disease post-surgery in CRC patients with liver metastases by quantitative monitoring of urinary ctDNA KRAS with single molecule sensitivity.

### Example 5. Early Detection of Responses to Chemotherapy in Patients with Metastatic Colorectal Cancer

A clinical problem with colorectal cancer (CRC) is that carcinoembryonic antigen (CEA) may be found at elevated levels in people colorectal cancer, but is unreliable as its levels are often inconsistent with CRC clinical outcomes. This study evaluates the use of urinary and plasma tumor markers for the early detection of response to chemotherapy in CRC.

Four (4) CRC patients positive for the KRAS mutation G12D or G13D in tissue were monitored for circulating tumor DNA (ctDNA) while on chemotherapy. Urine and plasma specimens were collected at baseline, at 2 weeks on treatment and then monthly. Urinary ctDNA was extracted using methods that preferentially isolate small fragmented DNA. That DNA was analyzed for ctDNA using PCR enrichment followed by NGS sequencing. Accurate quantitation was achieved by implemented standard curves with standardized reporting of number of KRAS copies per 100K genome equivalents (GE). The KRAS ctDNA mutation detection assay has sensitivity of 0.006% mutant copies in a background of wild-type DNA. The average total amount of ctDNA extracted from urine was 1470 ng (range, 95 to 13,966 ng); the average total amount of ctDNA extracted from plasma was 150 ng (range, 95 to 13,966 ng).

### Example Patient 1

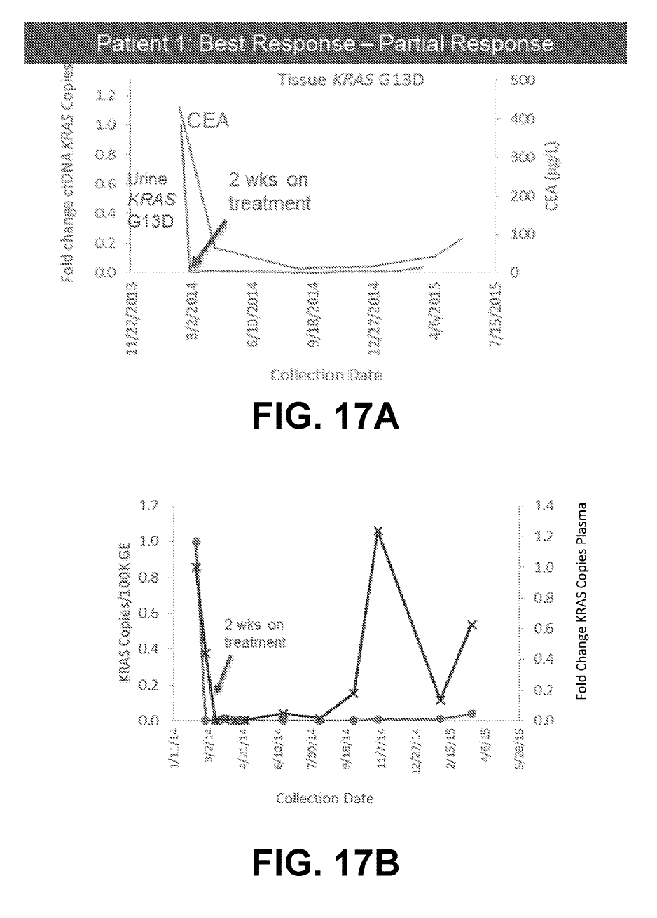

Patient 1 had metastatic disease to the liver, was treated with FOLFOX and had a partial response by imaging. The results in urine demonstrated that KRAS G13D burden decreased as early as 2 weeks on chemotherapy, consistent with a decrease in blood CEA concentration (FIG. 17A,B). This molecular response was detected in advance of imaging (earliest scan was done at 6 weeks). The patient subsequently started progressing. Importantly, 3 months prior to the CT scan that detected progression, a urine test detected an increase in the KRAS signal, thereby further demonstrating the value of monitoring for early signs of a progressive disease by urine.

### Example Patient 3

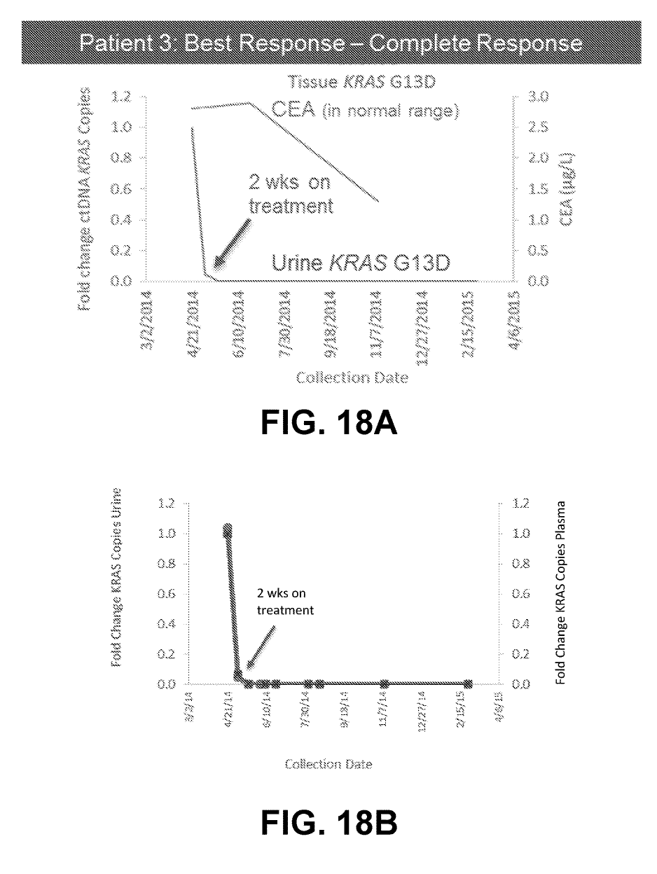

Patient 3 had liver metastases and was treated with neo-adjuvant FOLFOX followed by surgical removal of liver lesions. Surgery demonstrated complete pathological response. Urine testing predicted positive response to chemotherapy already at 2 weeks after beginning of therapy, while CEA levels were always in the normal range (below 3.5 μg/L) and were thus unusable (FIG. 18A). Urine and blood KRAS G13D levels were consistent with each other (FIG. 18B).

### Example Patient 6

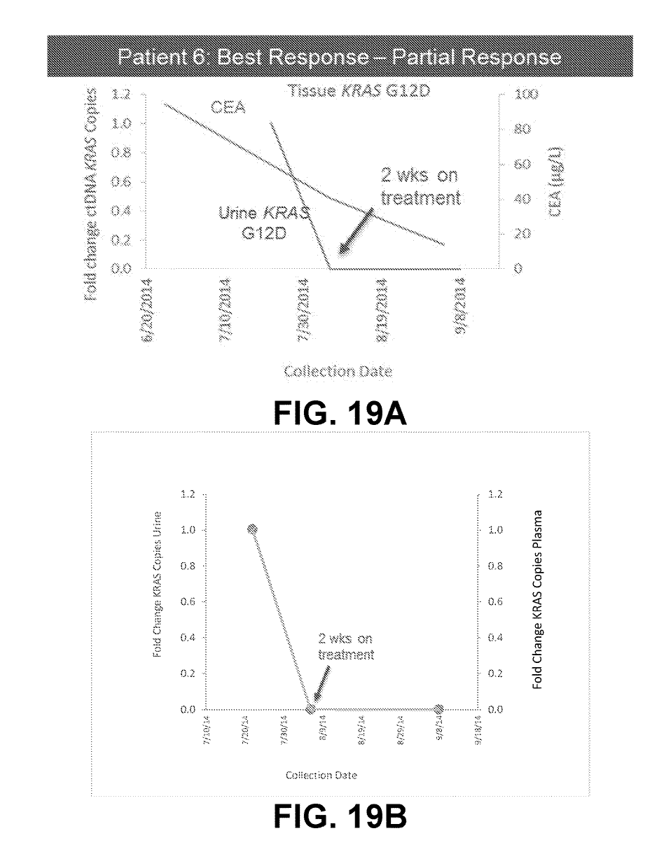

Patient 6 had liver metastasis and received FOLFOX. Imaging showed partial response to chemotherapy which was detected by both urine KRAS G12D testing at two weeks after treatment. CEA levels also predicted the response (FIG. 19A). Urine and plasma testing both predicted the response at two weeks (FIG. 19B).

### Example Patient 8

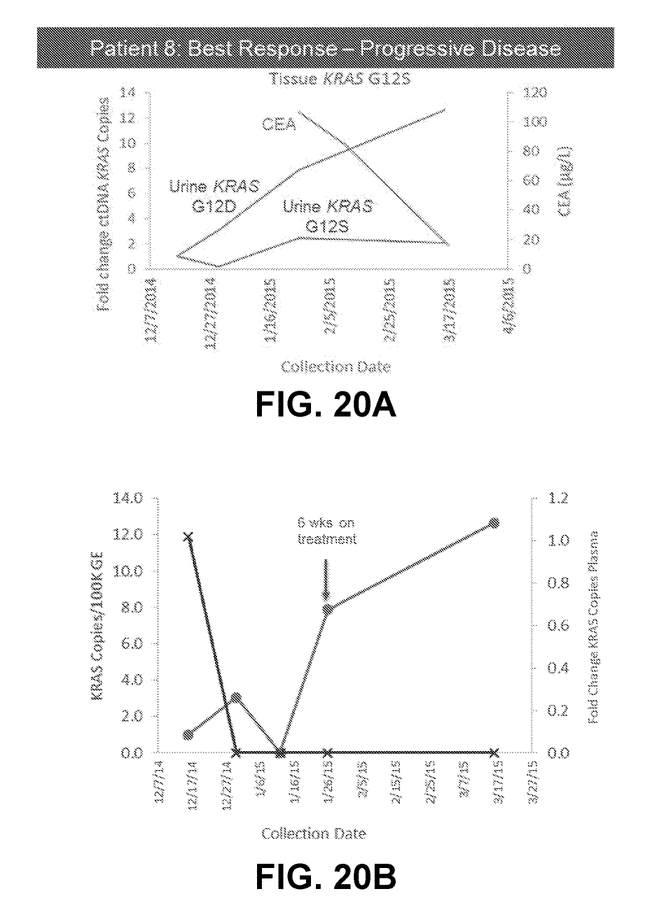

Patient 8 had both a primary tumor and a lung metastasis lesion. After surgical removal of the primary tumor in February, 2015, the patient was taken off chemotherapy for three months due to surgery. During that period of time, CT scans showed that the lung lesion was growing. As shown in FIG. 20A, the increase in urinary KRAS G12D paralleled that progression, but CEA levels appeared to decline, which appeared discordant with the clinical course. While the KRAS G12S mutation was determined in the primary tumor tissue, the predominant mutant, KRAS G12D, was undetected by tissue biopsy of the primary lesion. This clearly indicates heterogeneity between primary tumor and the metastatic lesion. Plasma testing did not detect the increase in KRAS G12D that was detected by urine testing (FIG. 20B).

### Conclusions

These results clearly demonstrate 100% concordance between urinary testing and the clinical course in metastatic CRC. This thus has immediate clinical utility for monitoring urinary KRAS in all CRC patients receiving chemotherapy. In this clinical setting, chemotherapy is typically given for 3-4 cycles followed by maintenance therapy (for example FOLFOX minus oxaliplatin). This goes in cycles and full chemo is re-introduced when patients are progressing by imaging. Maintenance therapy is less toxic, and detecting responses in this patients in advance of imaging will facilitate earlier transition to maintenance therapy and help alleviate the toxicities. Meanwhile, detecting early signs of progression will allow earlier re-introduction of aggressive chemotherapy regimens, all with the hope of a more effective, guided disease management.

### Example 6. Dynamics of KRAS G12/13 Allele Burden in ctDNA Predicts Survival in Patients with Unresectable Pancreatic Cancer Undergoing Palliative Chemotherapy

Median overall survival (OS) of patients with unresectable pancreatic cancer (PC) varies widely. Diagnostic tools are presently lacking to predict patient outcome or response to therapy. Further, imaging is not very accurate in reflecting tumor dynamics. The vast majority of pancreatic tumors harbor KRAS G12/13 mutations, which can be detected in circulating tumor (ct)DNA.

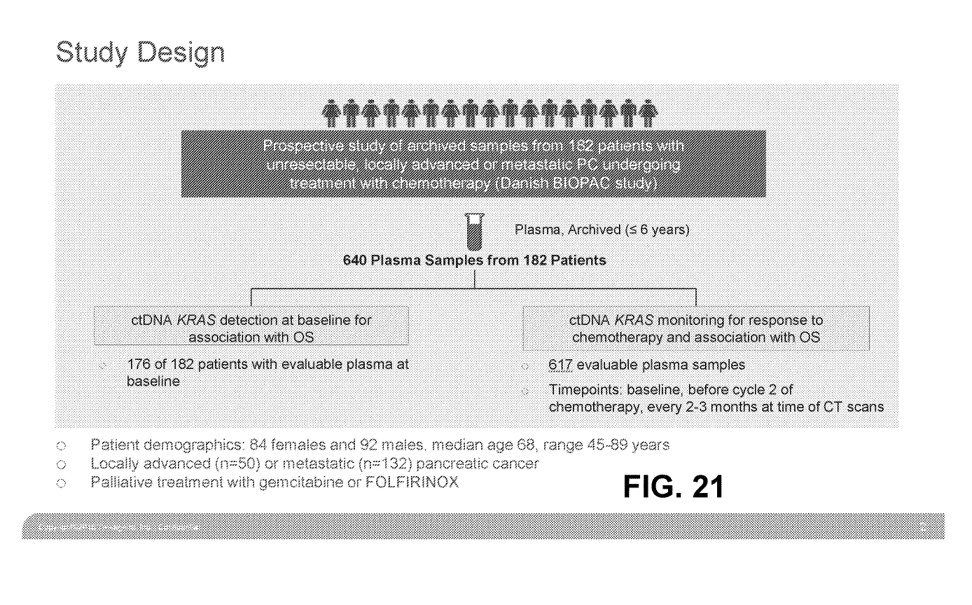

Results from prospective study with retrospectively analyzed archived samples from 182 patients with unresectable, locally advanced or metastatic pancreatic carcinoma (PC) undergoing treatment with chemotherapy (Danish BIOPAC study), provided here, demonstrates that high plasma KRAS G12/13 levels are prognostic for overall survival (OS). Furthermore, monitoring plasma KRAS G12/13 levels on chemotherapy improves predictive power of the baseline KRAS levels by taking into account the effect of treatment. FIG. 21 provides the study design.

Overall, study enrolled 1000 patients. There were 50 patients with loc advanced disease and 132 patients with metastatic disease. The investigation evaluated the association between baseline KRAS levels and overall survival as well as the dynamics of ctDNA KRAS in response to therapy and its association with OS.

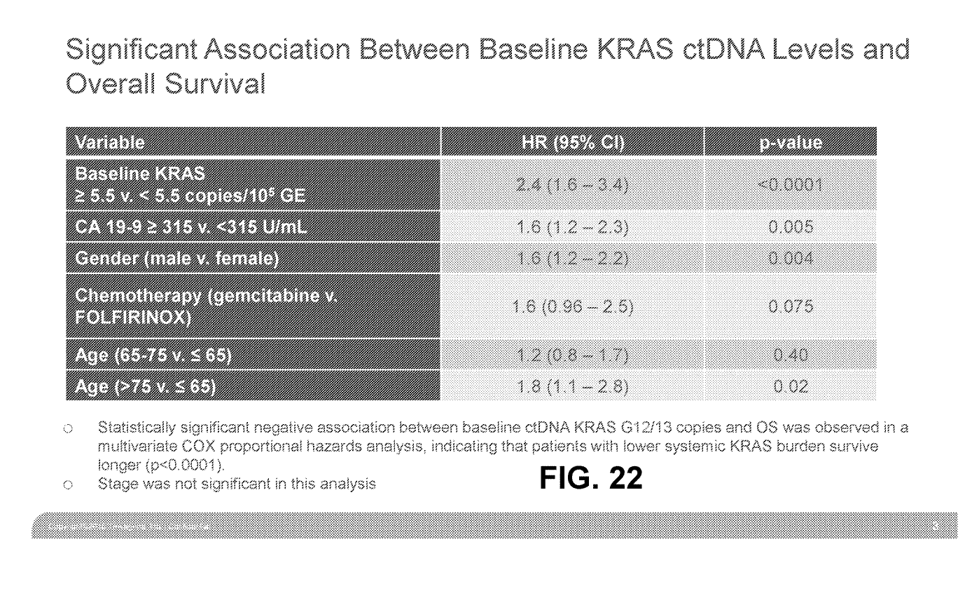

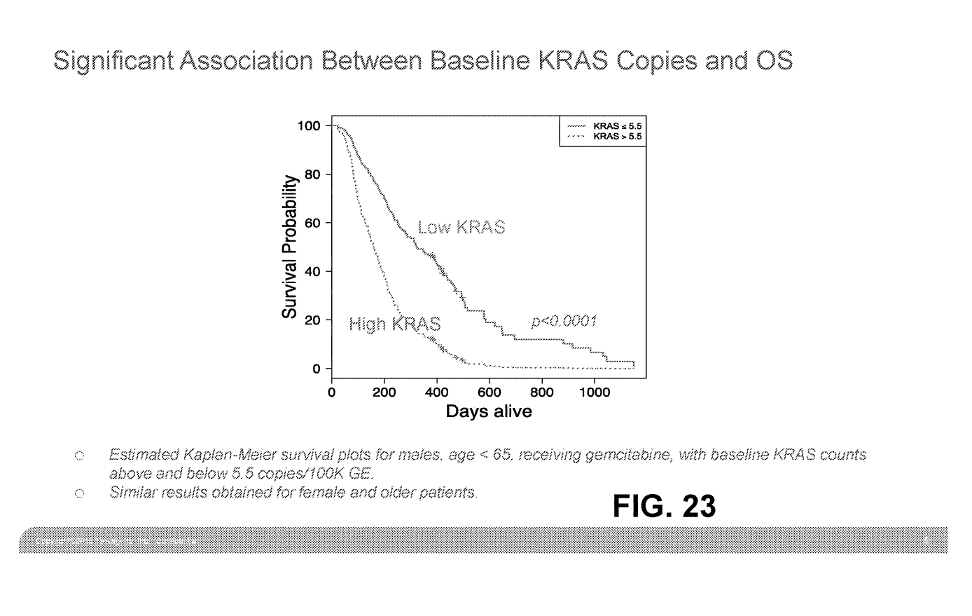

As shown in FIGS. 22 and 23, a multivariate analysis revealed a statistically significant negative association between baseline ctDNA KRAS G12/13 copies and OS, indicating that patients with lower systemic KRAS burden survive longer (p<0.0001). Ca-19-9 was an independent variable that also predicted survival. Gender was significant in this analysis, chemotherapy type was marginally significant; age was significant if we compared the groups older than 75 years old and younger than 65 years old. Stage was not significant in this analysis. The hazard ratio (HR) of death for patients with ≧5.5 KRAS copies/10⁵ genome equivalents (GE) is 2.4 times as high (95% CI: 2.0 to 4.9) as those with KRAS G12/13 copies <5.5/10⁵ GE.

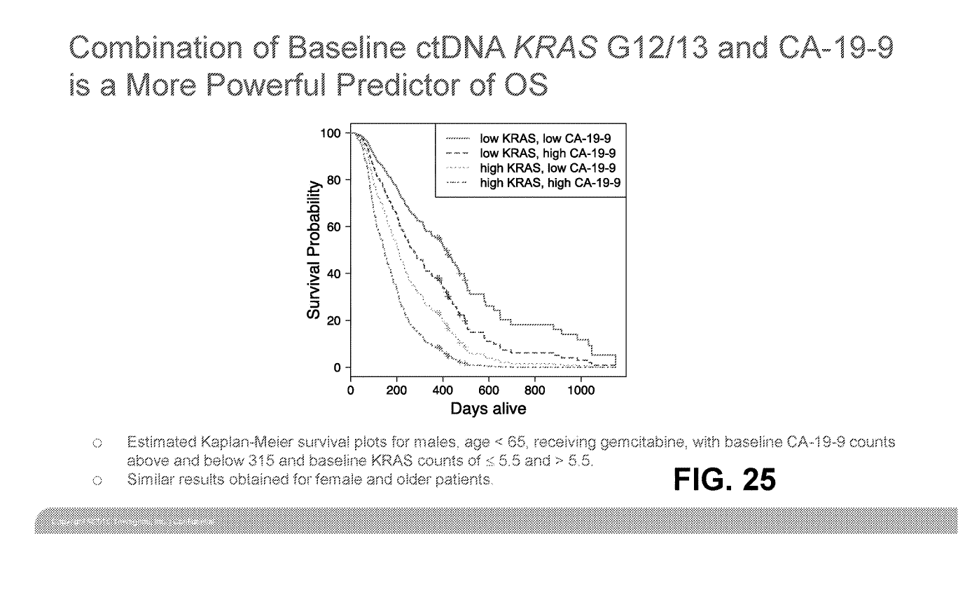

A combination of pre-treatment levels of ctDNA KRAS G12/13 and CA 19-9 demonstrated a stronger association with OS. (R2=23.9%, as compared 19.7% for the model with KRAS alone.) (FIGS. 24 and 25). HR of death for patients with ≧5.5 KRAS copies/105 GE and ≧315 U/mL CA 19-9 is 4.1 times as high as those with low KRAS and CA 19-9 (FIG. 24). In addition, combination of ctDNA KRAS and CA-19-9 identified a group of patients (17%) with significantly greater overall survival.

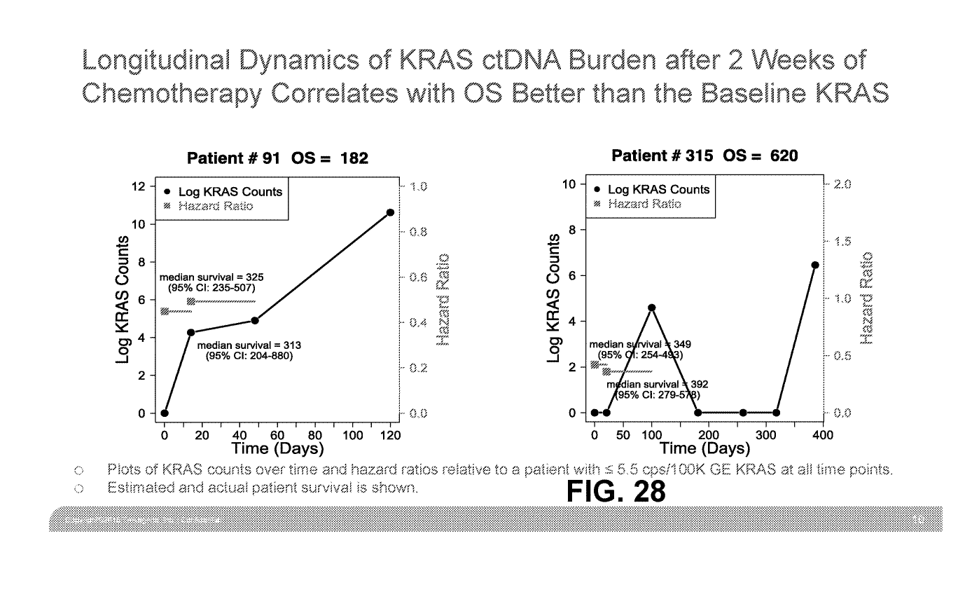

In order to account for the effect of therapy, a time-dependent model was built that allows adjustment of estimated patient survival based on the combination of pre-treatment ctDNA KRAS levels and KRAS levels after 2 weeks on first line chemotherapy. When taking into account ctDNA KRAS levels after 2 weeks on treatment, an estimated median survival more accurately reflects actual survival of individual patients (as compared to the median survival estimated based on pre-treatment ctDNA KRAS levels only) (FIGS. 26 and 27). Furthermore, we found that patients with decreases in ctDNA KRAS G12/13 within first 2 weeks of chemotherapy achieve survival benefits. For example, in the gemcitabine group, median survival of patients with high levels of KRAS before treatment was 148 days. If less than 100% decrease was observed after 2 weeks of therapy, patient survival did not improve (134 days). However, if KRAS levels decreased by 100% (became undetectable), patients achieved survival benefit and median survival was 224 days. Overall, our data suggest that monitoring ctDNA KRAS levels on therapy may reflect tumor response to therapy and better correlate with outcomes in patients with unresectable PC.

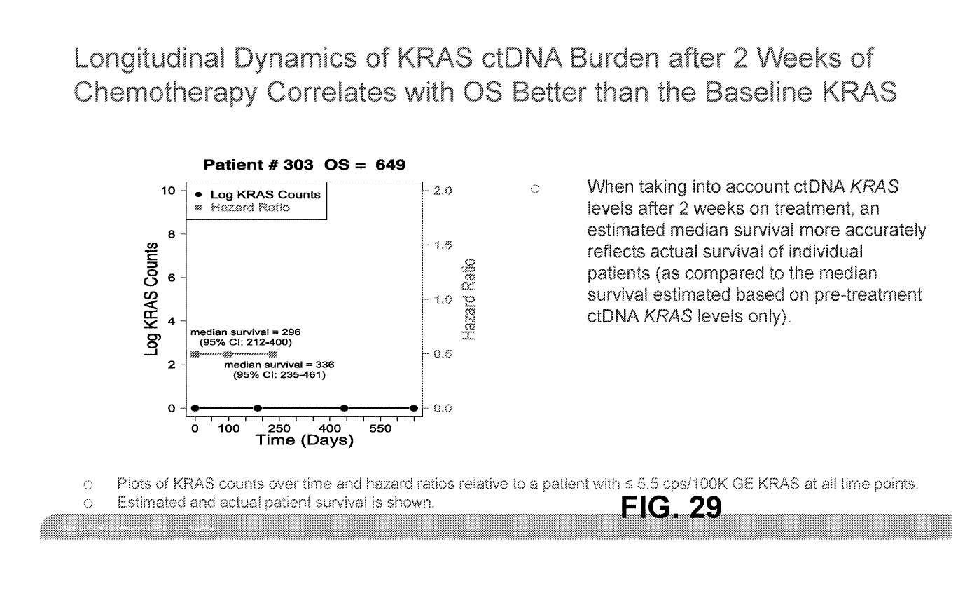

FIGS. 28 and 29 also show plots of KRAS counts over time and hazard ratios relative to a patient with ≦5.5 cps/100K GE KRAS at all time points. Estimated and actual patient survival is shown. These results show that, when ctDNA KRAS levels after 2 weeks on treatment are taken into account, the estimated median survival more accurately reflects actual survival of individual patients, when as compared to the median survival estimated based on pre-treatment ctDNA KRAS levels only.

### CONCLUSIONS

There was a significant negative association between baseline ctDNA KRAS counts and OS (p<0.0001), indicating that patients with lower KRAS burden in ctDNA survive longer.

Additionally, the combination of pre-treatment levels of KRAS and CA 19-9 was a better predictor of overall survival than either marker alone.

Further, patients with decreases in ctDNA KRAS levels on chemotherapy after 2 weeks of treatment achieved survival benefit. Also, the combination of baseline ctDNA KRAS burden and KRAS levels after 2 weeks on chemotherapy was a better predictor of patient outcomes than baseline ctDNA KRAS alone.

Based on the above, monitoring ctDNA KRAS dynamics appears to be clinically useful for treatment management decisions in non-resectable patients with pancreatic cancer.

### REFERENCES

Bettegowda et al., 2014, Sci Transl Med 6:224ra224.
Buffery, D., 2015, Am Health Drug Benefits 8:216-222.
Cross et al., 2014, Cancer Discov 4:1046-1061.
Crowley et al., 2013, Nat. Rev. Clin. Oncol. 10:472-484.
Dawson et al., 2013, N Engl J Med 368, 1199-1209.
Diaz, L. A., Jr. & Bardelli, 2014, J Clin Oncol 32:579-586.
Diaz et al., 2012, Nature 486:537-540.
Eisenhauer et al., 2009, Eur. J. Cancer 45:228-247.
Gainor et al., 2014, Clin. Cancer Res. 20:2587-2594.
Haber, D. A. & Velculescu, V. E., 2014, Cancer Discov 4:650-661.
Hyman et al., 2015, Cancer Discov 5:64-71.
Jahr et al., 2001, Cancer Res 61:1659-1665.
Janku et al., 2014, Oncotarget 5, 3607-3610 (2014).
Janne et al., 2015, N Engl J Med 372:1689-1699.
Karachaliou et al., 2015, JAMA Oncol 1:149-157.
Kuang et al., 2009, Clin. Cancer Res. 15:2630-2636.
Kurzrock et al., 2013, Ann Oncol 24: 2256-2261.
Leary et al., 2012, Sci Transl Med 4:162ra154.
Marchetti et al., 2015, J Thorac Oncol 10:1437-1443.
Melkonyan et al., 2008, Ann N Y Acad Sci 1137:73-81.
Neal et al., 2014, Nat. Rev. Clin. Oncol. doi: 10.1038/nrclinonc.2014.222. [Epub ahead of print].
Newman et al., 2014, Nat Med 20:548-554.
Piotrowska et al., 2015, Cancer Discov 5:713-722.
Poole et al., November 2014, Poster P143, EORTC-AACR meeting.
Poole et al., January 2015, Poster C46, ASCO-GI meeting.
Schwarzenbach et al., Nat Rev Cancer 11:426-437.
Siravegna et al., 2015, Nat Med 21:795-801.
Su et al., 2004, J Mol Diagn 6:101-107.
Thress et al., 2015, Nat Med 21, 560-562.
PCT Patent Application WO 2015/073163.
PCT Patent Publication WO 2015/058176.
US Patent Publication US 2010/0068711.
US Patent Publication US 2013/0064789.
US Patent Publication US 2013/0130264.
U.S. Pat. RE39920E1.

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

What is claimed is:

1. A method comprising quantifying a mutation in cell-free DNA in a plurality of samples of a bodily fluid of a subject, each sample taken at a different time point after the subject begins a treatment,
wherein the mutation is associated with a cancer in the subject, and the treatment is against the cancer, and
wherein a sample is taken prior to, or at, the beginning of the treatment, and within seven days after beginning the treatment.

2. The method of claim 1, wherein a sample is taken prior to, or at, the beginning of the treatment, and at least twice within seven days after beginning the treatment.

3. The method of claim 1, wherein a sample is taken daily for seven days after beginning the treatment.

4-15. (canceled)

16. The method of claim 1, wherein the mutation is a resistance mutation that was acquired after a first treatment for a cancer with a different mutation.

17. (canceled)

18. The method of claim 16, wherein the resistance mutation is EGFR T790M, an ALK mutation, a ROS1 mutation, or a RET mutation.

19. The method of claim 1, wherein the mutation is the original mutation of the cancer.

20. The method of claim 1, wherein the mutation is a point mutation, insertion, deletion, indel, or rearrangement.

21. The method of claim 1, wherein the mutation associated with the cancer is a point mutation in an ABL1, BRAF, CHEK1, FANCC, GATA3, JAK2, MITF, PDCD1LG2, RBM10, STAT4, ABL2, BRCA1, CHEK2, FANCD2, GATA4, JAK3, MLH1, PDGFRA, RET, STK11, ACVR1B, BRCA2, CIC, FANCE, GATA6, JUN, MPL, PDGFRB, RICTOR, SUFU, AKT1, BRD4, CREBBP, FANCF, GID4(C17orf39), KAT6A (MYST3), MRE11A, PDK1, RNF43, SYK, AKT2, BRIP1, CRKL, FANCG, GLI1, KDM5A, MSH2, PIK3C2B, ROS1, TAF1, AKT3, BTG1, CRLF2, FANCL, GNA11, KDM5C, MSH6, PIK3CA, RPTOR, TBX3, ALK, BTK, CSF1R, FAS, GNA13, KDM6A, MTOR, PIK3CB, RUNX1, TERC, AMER1 (FAM123B), C11orf30 (EMSY), CTCF, FAT1, GNAQ, KDR, MUTYH, PIK3CG, RUNX1T1, TERT promoter, APC, CARD11, CTNNA1, FBXW7, GNAS, KEAP1, MYC, PIK3R1, SDHA, TET2, AR, CBFB, CTNNB1, FGF10, GPR124, KEL, MYCL (MYCL1), PIK3R2, SDHB, TGFBR2, ARAF, CBL, CUL3, FGF14, GRIN2A, KIT, MYCN, PLCG2, SDHC, TNFAIP3, ARFRP1, CCND1, CYLD, FGF19, GRM, 3 KLHL6, MYD88, PMS2, SDHD, TNFRSF14, ARID1A, CCND2, DAXX, FGF23, GSK3B, KMT2A (MLL), NF1, POLD1, SETD2, TOP1, ARID1B, CCND3, DDR2, FGF3, H3F3A, KMT2C (MLL3), NF2, POLE, SF3B1, TOP2A, ARID2, CCNE1, DICER1, FGF4, HGF, KMT2D (MLL2), NFE2L2, PPP2R1A, SLIT2, TP53, ASXL1, CD274, DNMT3A, FGF6, HNF1A, KRAS, NFKBIA, PRDM1, SMAD2, TSC1, ATM, CD79A, DOT1L, FGFR1, HRAS, LMO1, NKX2-1, PREX2, SMAD3, TSC2, ATR, CD79B, EGFR, FGFR2, HSD3B1, LRP1B, NOTCH1, PRKAR1A, SMAD4, TSHR, ATRX, CDC73, EP300, FGFR3, HSP9OAA1, LYN, NOTCH2, PRKCI, SMARCA4, U2AF1, AURKA, CDH1, EPHA3, FGFR4, IDH1, LZTR1, NOTCH3, PRKDC, SMARCB1, VEGFA, AURKB, CDK12, EPHA5, FH, IDH2, MAGI2, NPM1, PRSS8, SMO, VHL, AXIN1, CDK4, EPHA7, FLCN, IGF1R, MAP2K1, NRAS, PTCH1, SNCAIP, WISP3, AXL, CDK6, EPHB1, FLT1, IGF2, MAP2K2, NSD1, PTEN, SOCS1, WT1, BAP1, CDK8, ERBB2, FLT3, IKBKE, MAP2K4, NTRK1, PTPN11, SOX10, XPO1, BARD1, CDKN1A, ERBB3, FLT4, IKZF1, MAP3K1, NTRK2, QKI, SOX2, ZBTB2, BCL2, CDKN1B, ERBB4, FOXL2, IL7R, MCL1, NTRK3, RAC1, SOX9, ZNF217, BCL2L1, CDKN2A, ERG, FOXP1, INHBA, MDM2, NUP93, RAD50, SPEN, ZNF703, BCL2L2, CDKN2B, ERRFI1, FRS2, INPP4B, MDM4, PAK3, RAD51, SPOP, BCL6, CDKN2C, ESR1, FUBP1, IRF2, MED12, PALB2, RAF1, SPTA1, BCOR, CEBPA, EZH2, GABRA6, IRF4, MEF2B, PARK2, RANBP2, SRC, BCORL1, CHD2, FAM46C, GATA1, IRS2, MEN1, PAX5, RARA, STAG2, BLM, CHD4, FANCA, GATA2, JAK1, MET, PBRM1, RB1, or STAT3 gene, or a rearrangement in an ALK, BRAF, BRD4, ETV4, FGFR1, KIT, MYC, NTRK2, RARA, TMPRSS2, BCL2, BRCA1, EGFR, ETV5, FGFR2, MSH2, NOTCH2, PDGFRA, RET, BCR, BRCA2, ETV1, ETV6, FGFR3, MYB, NTRK1, RAF1, or ROS1 gene.

22. The method of claim 1, wherein the mutation associated with the cancer is in an APC, ALK, BRAF, CDK4, CTNNB1, EGFR, FGFR1, FGFR2, FGFR3, HER3, PDGFRA, PDGFRB, AKT1, ESR1, AR, EZH2, FLT3, HER2, IDH1, IDH2, JAK2, KIT, KRAS, c-Myc, MEK1, NOTCH1, NRAS, PIK3CA, PTEN, SNV, TP53, CDKN2A, or RB1 gene.

23. The method of claim 1, wherein the mutation associated with the cancer is in the EGFR gene.

24. The method of claim 23, wherein the EGFR mutation is an EGFR activing mutation.

25. The method of claim 23, wherein the mutation is EGFR T790M, L858R or Exon 19del.

26. The method of claim 1, wherein the cancer is a lung cancer, colorectal cancer, or pancreatic cancer.

27. The method of claim 1, wherein the mutation associated with the cancer is in the KRAS gene.

28. The method of claim 27, wherein the KRAS mutation is KRAS G12D, G12S, or G13D.

29-40. (canceled)

41. The method of claim 1, wherein the bodily fluid is peripheral blood, serum, plasma, or urine.

42. The method of claim 1, wherein the bodily fluid is urine.

43-44. (canceled)

45. The method of claim 1, wherein the treatment comprises chemotherapy, radiation therapy, surgery, hormone therapy, therapy targeting a particular cancer gene or pathway (“targeted therapy”), immunotherapy, or photodynamic therapy.

46. The method of claim 1, wherein the treatment comprises targeted therapy.

47. The method of claim 41, wherein the targeted therapy is administration of a tyrosine kinase inhibitor, a serine/threonine kinase inhibitor, compound targeting CD20, Her2/neu, the folate receptor, EGFR, PDGFR, KIT, VEGFR2 or a VEGF ligand.

48-74. (canceled)
